(19) United States
(12) Patent Application Publication
(10) Pub. No.: US 2026/0243770 A1
(43) Pub. Date: Aug. 20, 2026

(54) Methods And Uses Involving Aquaporin-5 (AQP5)

(71) Applicant: AGENCY FOR SCIENCE, TECHNOLOGY AND RESEARCH, Singapore (SG)

(72) Inventors: Nicholas Barker, Singapore (SG); Grace Lim, Singapore (SG); Swathi Yada, Singapore (SG); Si Hui Tan, Singapore (SG)

(21) Appl. No.: 19/165,205

(22) PCT Filed: Mar. 14, 2024
(86) PCT No.: PCT/SG2024/050154

§ 371 Date: Sep. 15, 2025

(30) Foreign Application Priority Data
Mar. 14, 2023 (SG) 10202300699X
Sep. 27, 2023 (SG) 10202302747V

(51) Int. Cl. G01N 33/5753; C12N 15/113; C12Q 1/6886; G01N 33/58

(52) U.S. Cl. CPC G01N 33/5753; C12N 15/113; C12Q 1/6886; G01N 33/582; C12N 2310/14; C12Q 2600/158

(57) ABSTRACT

Disclosed herein are methods of identifying a gastric cancer stem cell or gastric cancer stem cell population. Also described herein are methods of isolating one or more gastric cancer stem cells from a cell population, comprising contacting cells of the cell population with an agent that binds to AQP5, isolating one or more AQP5-expressing cells that are bound to the agent, wherein the one or more AQP5-expressing cells are gastric cancer stem cells. Further described herein are methods of ablating or eliminating an AQP5+ gastric cancer stem cells, as well as methods of treating gastric cancer, and methods of monitoring gastric cancer tumorigenesis or progression of gastric cancer.

### CROSS-REFERENCE TO RELATED APPLICATIONS

This application claims the benefit of priority of Singapore provisional application no. 10202300699X, filed 14 Mar. 2023, and Singapore provisional application no. 10202302747V, filed 27 Sep. 2023, the contents of it being hereby incorporated by reference in its entirety for all purposes.

### FIELD OF THE INVENTION

The present invention relates generally to the field of molecular biology. In particular, the present invention relates to the use of biomarkers for the detection and diagnosis of cancer.

### BACKGROUND OF THE INVENTION

Gastric cancer is one of the leading causes of cancer-related deaths worldwide and in Singapore, with a five-year survival rate of less than 30%. Treatment of gastric cancer is currently limited to traditional methods of chemotherapy, radiation therapy, and surgical resection, but these approaches remain ineffective in ameliorating cancer relapse in many gastric cancer patients, particularly for diffuse-type gastric cancers. Treatment-resistant cancer stem cell populations are considered to be critical drivers of gastric cancer relapse. Yet, proposed gastric cancer stem cell markers to date are either lacking robust functional validations directly demonstrating the stem potential of the labelled cell populations, or are broadly expressed across a wide range of normal and tumour tissues, making it challenging to target these cells in patients.

The implementation of the concept of targeting cancer stem cells in the treatment of various human cancers in the context of gastric cancer had previously been hindered by a paucity of gastric cancer stem cell markers, most of which had not been robustly validated using near-physiological cancer models and functional assays. Moreover, some previously proposed markers are broadly expressed across both normal and tumour tissues, making it challenging to develop these markers into therapeutic targets safe for patients.

There is thus an unmet need for methods of identifying gastric cancer stem cells.

### SUMMARY

In one aspect, the present disclosure refers to a method of identifying a gastric cancer stem cell or gastric cancer stem cell population comprising a1) detecting expression of Aquaporin 5 (AQP5) in a cell or cell population; or b1) detecting expression level of AQP5 in a cell or a cell population and comparing the expression level with the expression level of AQP5 in a reference cell or reference cell population, wherein detection of AQP5 expression in the cell or cell population, or expression of AQP5 in the cell or cell population at an increased level compared to the reference cell or reference cell population identifies said cell or cell population as a gastric cancer stem cell or gastric cancer stem cell population.

In another aspect, the present disclosure refers to a method of isolating one or more gastric cancer stem cells from a cell population, comprising i) contacting cells of the cell population with an agent that binds to AQP5; ii) isolating one or more AQP5-expressing cells that are bound to the agent, wherein the one or more AQP5-expressing cells are gastric cancer stem cells.

In yet another aspect, the present disclosure refers to a method of ablating or eliminating an AQP5+ gastric cancer stem cell that has been modified to express the DTR gene or an inducible Caspase9 (iCasp9) gene, comprising contacting said cell with a diphtheria toxin (DT) or an inducer of iCasp9.

In a further aspect, the present disclosure refers to a method of ablating or eliminating an AQP5+ gastric cancer stem cell comprising contacting the cell with an agent that binds to AQP5, wherein binding of the AQP5+ cell with the agent ablates or eliminates the cell.

In one aspect, the present disclosure refers to a method of treating gastric cancer in a subject in need thereof, comprising administering a therapeutically effective amount of one or more agents that eliminates or ablates AQP5-expressing cells to the subject.

In another aspect, the present disclosure refers to a biomarker of gastric cancer stem cells, wherein the biomarker is AQP5.

In a further aspect, the present disclosure refers to a kit for identifying, isolating, eliminating or ablating a gastric cancer stem cell, comprising an agent that binds to AQP5, and instructions for use.

In yet another aspect, the present disclosure refers to a method of inhibiting gastric cancer tumorigenesis or gastric cancer progression, the method comprising administering an AQP5 inhibitor to a subject.

In another aspect, the present disclosure refers to a method of promoting gastric cancer regression, the method comprising administering an AQP5 inhibitor to a subject.

In one aspect, the present disclosure refers to a method of monitoring progression of a gastric cancer in a subject, the method comprising: a) measuring the expression level of AQP5 in a sample obtained from the subject after having undergone treatment for gastric cancer; and b) measuring the expression level of AQP5 in a control sample obtained from the subject prior to treatment for gastric cancer; wherein an increase in the expression level of AQP5 in the sample of step a compared to the control sample indicates that gastric cancer tumorigenesis has taken place or that the gastric cancer has progressed.

In another aspect, the present disclosure refers to a method of monitoring gastric cancer tumorigenesis in a subject, the method comprising: c) measuring the expression level of AQP5 in a sample obtained from the subject; and d) measuring the expression level of AQP5 in a reference sample obtained from the subject at a timepoint earlier than the sample of step c; wherein an increase in the expression level of AQP5 in the sample of step c compared to the reference sample of step d indicates that gastric cancer tumorigenesis has taken place.

### BRIEF DESCRIPTION OF THE DRAWINGS

The invention will be better understood with reference to the detailed description when considered in conjunction with the non-limiting examples and the accompanying drawings, in which:

FIG. 1 shows results indicating that Aqp5 is overexpressed in mouse and human pyloric tumours. FIG. 1A shows a schematic of the Aqp5-eGFPires-CreERT2; Apc^{fl/fl}; Pten^{fl/fl}; Kras^{LSL-G12D/+}; Rosa26-tdTomato^{LSL} conditional genetic mouse model for pyloric cancer. FIG. 1B shows fluorescent images denoting AQP5 expression in the pylorus across multiple stages of tumour development in the Aqp5-Cre/APK mouse model. FIG. 1C shows a bar graph quantification of the changes in proportion of AQP5+ cells within the healthy and tumour pyloric tissues over time. FIG. 1D shows the FACS gating strategy used for isolation of tumour epithelial (dTom+) Aqp5− and Aqp5+ cells from primary mouse pyloric tumours. FIG. 1E shows a bar graph depicting the relative Aqp5 expression in sorted mouse tumour cell populations by qPCR. FIG. 1F is a heatmap showing the top 50 differentially expressed genes (DEGs) between Aqp5+ and Aqp5− samples isolated from 6 independent mouse pyloric tumours. FIG. 1G shows slides indicating AQP5 expression in human gastric tissue microarrays of the normal stomach, intestinal-type gastric tumours, diffuse-type gastric tumours, and mixed-type gastric tumours. Staining was scored on a scale ranging from absence of staining to strong staining as indicated in the legend (right). Images shown are representative of tissue cores presenting moderate staining (in the normal stomach) and strong staining (in the tumour tissues). FIG. 1H shows the FACS gating strategy used for isolation of tumour epithelial Aqp5− and Aqp5+ cells from primary human pyloric tumour biopsies. FIG. 1I shows a bar graph depicting the relative Aqp5 expression in sorted human tumour cell populations by qPCR. FIG. 1J is a heatmap of the top 50 DEGs between Aqp5+ and Aqp5− samples isolated from 5 independent human pyloric tumours. Scale bars, 200 μm. Graphs represent mean±S.D. with two-tailed unpaired t-test.

FIG. 2 shows results indicating that Mouse and human Aqp5+ pyloric tumour cells function as cancer stem cells. FIG. 2A shows images of organoids generated from single FACS-sorted mouse Aqp5+ and Aqp5− cells at passages 0, 2, and 10. Mouse Aqp5+ cell-derived organoids maintain high outgrowth efficiency until at least P10, while the small number of Aqp5-cell-derived organoids that form fail to survive beyond P2. FIG. 2B shows a bar graph showing data indicating organoid outgrowth efficiency of single mouse Aqp5+ and Aqp5− cells at P0 and quantification of highest passage number reached for each culture. FIG. 2C shows fluorescent images denoting the expression of AQP5 and differentiated lineage markers KI67, TFF2, and CHGA in mouse Aqp5+ cell-derived organoids. FIG. 2D shows whole mount and H&E images of tumours generated from transplanted mouse Aqp5+ (left) and Aqp5-(right) pyloric tumour cells. FIG. 2E shows a bar graph quantification of tumour volumes from transplanted Aqp5+ and Aqp5-pyloric tumour cells. FIG. 2F shows images of organoids generated from single FACS-sorted human Aqp5+ and Aqp5− cells at passages 0, 2, and 10. Human Aqp5+ cell-derived organoids maintain high outgrowth efficiency until at least P10, while the small number of Aqp5-cell-derived organoids fail to survive beyond P2. FIG. 2G is a bar graph showing organoid outgrowth efficiency of single human Aqp5+ and Aqp5-cells at P2 and quantification of highest passage number reached for each culture. FIG. 2H are fluorescent images denoting the expression of AQP5 and differentiated lineage markers KI67, MUC5AC, and CHGA in human Aqp5+ cell-derived organoids. Scale bars, 500 μm (FIG. 2A, FIG. 2F) and 100 μm (FIG. 2C, FIG. 2H). Graphs represent mean±S.D. with two-tailed unpaired t-test.

FIG. 3 shows results indicating that Ablation of Aqp5+ cancer stem cells blocks tumour initiation and progression. FIG. 3A shows images showing the results of diphtheria toxin (DT) treatment of Aqp5-Cre/APK/DTR mouse pyloric tumour organoids (with Aqp5-2A-DTR allele integrated) during organoid initiation (day 0) blocks organoid outgrowth. n=4 biological replicates. FIG. 3B is a line graph showing the quantification of changes in organoid numbers over time in control and DTtreated organoid cultures following treatment on day 0. FIG. 3C shows images depicting the results of diphtheria toxin (DT) treatment of Aqp5-Cre/APK/DTR mouse pyloric tumour organoids during organoid growth/maintenance (day 3) results in rapid decline of the cultures. n=4 biological replicates. FIG. 3D shows a line graph depicting the quantification of changes in organoid numbers over time in control and DT-treated organoid cultures following treatment on day 3. FIG. 3E shows images showing that intraperitoneal DT administration to mice orthotopically transplanted with Aqp5-Cre/APK/DTR organoids during early stages of tumour initiation (day 3) results in complete absence of tumour outgrowth. n=5 biological replicates. FIG. 3F shows images showing that intraperitoneal DT administration to mice orthotopically transplanted with Aqp5-Cre/APK/DTR organoids at later stages of tumour growth (week 4) drives tumour regression. n=5 biological replicates. g, Strategy for Aqp5+ cell ablation within human gastric cancer organoids. The Aqp5-2A-iCaspase9 cassette is integrated into human gastric cancer organoids to drive expression of inducible Caspase9 within Aqp5+ cells. Addition of B/B Homodimerizer promotes dimerization of Caspase9 and activation of apoptotic pathways leading to cell death. FIG. 3H shows images depicting results of dimerizer (Dim) treatment of Aqp5-2A-iCaspase9 human gastric cancer organoids hinders organoid growth in vitro. FIG. 3I shows a bar graph quantification of organoid numbers in control and dimerizer-treated cultures. Scale bars, 500 μm (FIG. 3A, FIG. 3C, FIG. 3G) and 1 mm (FIG. 3E, FIG. 3F). Graphs represent mean±S.D. with two-tailed unpaired t-test.

FIG. 4 shows results indicating that Aqp5 is expressed in a subset of cells within mouse pyloric tumours. FIG. 4A shows representative whole mount and H&E images of an Aqp5-Cre/APK mouse pyloric tumour. FIG. 4B shows images of the results of RNAscope of Aqp5 and Lgr5 in the healthy mouse pylorus, showing their co-localization within the pyloric stem cell compartment in gland bases. FIG. 4C shows a schematic of the Aqp5-2ACreERT2; Apcfl/fl; Ptenfl/fl; KrasLSL-G12D/+; Rosa26-tdTomatoLSL conditional genetic mouse model for pyloric cancer (left). AQP5 levels increase over time during tumour development and resembles expression patterns of tumours derived from Aqp5-eGFP-ires-CreERT2 models (right). FIG. 4D shows images of MERSCOPE-based spatial transcriptomics of a Aqp5-Cre/APK mouse pyloric tumour section revealing highly heterogenous cell types assigned by Leiden clustering, with the Aqp5-expressing clusters highlighted. FIG. 4E shows a UMAP plot of Aqp5-Cre/APK tumour cell clusters. FIG. 4F shows a heatmap of expression levels of selected genes in each of the 8 epithelial cell clusters showing heterogeneity of Aqp5+ populations relative to other tumour cell lineages. FIG. 4G shows images of RNAscope of Aqp5 and putative gastric cancer stem cell markers Lgr5, Cxcr4, and Cd44 within Aqp5-Cre/APK mouse pyloric tumour sections. Aqp5 expression overlaps partially with known stem markers and also mark unique tumour compartments. Scale bars, 50 μm (b, g—zoomed insets) and 200 μm (FIG. 4A, FIG. 4C, FIG. 4D, FIG. 4G).

FIG. 5 shows results of transcriptomic analyses of mouse Aqp5+ and Aqp5− pyloric tumour cells. FIG. 5A shows bar graphs depicting selected gene ontology (GO) terms from the molecular function (MF) and biological process (BP) categories that were significantly enriched in mouse Aqp5+ cells compared to Aqp5− cells. FIG. 5B shows results of a gene set enrichment analysis (GSEA) of the epithelial-mesenchymal transition and external encapsulating structure organization pathways (left), with accompanying heat maps showing relative expression of selected genes within each pathway (right). FIG. 5C to FIG. 5F shows the results of qPCR validation (left graphs) and RNAseq expression data (right graphs) of selected genes upregulated in the mouse Aqp5+ cell transcriptome relating to the tumour microenvironment (FIG. 5C), metastatic dissemination (FIG. 5D), gastric cancer progression (FIG. 5E), and drug resistance (FIG. 5F). FIG. 5G shows images of RNAscope validation of selected targets Lgr5, Pthlh, Rgs5, and Hey1 confirming their upregulation in Aqp5+ tumour cell regions. Scale bars, 100 μm. Graphs represent mean±S.D. with two-tailed unpaired t-test.

FIG. 6 shows results indicting that Aqp5 is expressed in a subset of cells within human pyloric tumours. FIG. 6A show a UMAP plot of cell clusters generated from scRNAseq of healthy and tumour human gastric tissues, with the epithelial cell clusters labelled. FIG. 6B shows a list of selected markers for assigning identities to the epithelial cell clusters. FIG. 6C shows a UMAP plot showing Aqp5 expression in the epithelial cell clusters within normal (left) and tumour (right) human gastric cells. FIG. 6D shows a Violin plot of Aqp5 expression in each of the epithelial clusters showing elevated levels within tumour cell populations. FIG. 6E to FIG. 6F shows UMAP plots (FIG. 6E) and violin plots (FIG. 6F) of tumour epithelial cell clusters separated by tumour subtype: intestinal, diffuse, and mixed. FIG. 6G to FIG. 6H shows UMAP plots (FIG. 6G) and violin plots (FIG. 6H) of tumour epithelial cell clusters separated by tumour location: antrum/pylorus, body, and cardia. FIG. 6I shows an UMAP plot of epithelial cell clusters generated from scRNAseq of healthy and tumour human gastric tissues from a second dataset GSE150290. FIG. 6J shows the identification of elected markers for assigning identities to the epithelial cell clusters in FIG. 6I. FIG. 6K shows a violin plot of Aqp5 expression in each of the epithelial clusters identified in FIG. 6I showing elevated levels within tumour populations.

FIG. 7 shows the results of transcriptomic analyses of human Aqp5+ and Aqp5− pyloric tumour cells. FIG. 7A shows bar graphs depicting selected gene ontology (GO) terms from the molecular function (MF) and biological process (BP) categories that were significantly enriched in human Aqp5+ cells compared to Aqp5− cells. FIG. 7B shows results of a gene set enrichment analysis (GSEA) of the epithelial-mesenchymal transition and external encapsulating structure organization pathways (left), with accompanying heat maps showing relative expression of selected genes within each pathway (right). FIG. 7C to FIG. 7F shows the results of qPCR validation (left graphs) and RNAseq expression data (right graphs) of selected genes upregulated in the human Aqp5+ cell transcriptome relating to the tumour microenvironment (FIG. 7C), cancer progression (FIG. 7D), drug resistance (FIG. 7E), and tumour regulation (FIG. 7F). FIG. 7G shows results of RNAscope validation of selected targets AQP5, CLDN2, DCHS2, HEY2, and PLA1A confirming their upregulation in Aqp5+ tumour cell regions. Scale bars, 100 μm. Graphs represent mean±S.D. with two-tailed unpaired t-test.

FIG. 8 shows results indicating that mouse Aqp5+ cells function as cancer stem cells. FIG. 8A shows representative brightfield and immunofluorescence images of mouse Aqp5− Cre/APK pyloric tumour organoids generated from whole glands showing comparable organoid morphology and expression of gastric lineage markers as tumour organoids formed from isolated Aqp5+ tumour cells. FIG. 8B shows representative whole mount, H&E and IHC images of the stomach of an immunedeficient NOG mouse 4 months following orthotopic transplantation of primary mouse pyloric tumour cells into the gastric submucosa, with minimal tumour cell establishment. FIG. 8C shows a schematic of the experimental workflow for generating orthotopic tumours from transplantation of mouse pyloric tumour organoids, isolating Aqp5+ and Aqp5− cells by FACS, and re-transplanting these cells into immune-deficient mice. FIG. 8D shows images showing the histology of mouse Aqp5-Cre/APK pyloric tumour organoids as assessed by H&E (left) and the orthotopic tumours generated from transplanting these tumour organoids visualised by whole mount and H&E (centre and right). FIG. 8E shows the FACS gating strategy for isolating transplanted tumour epithelial (dTom+) Aqp5+ and Aqp5− cells. FIG. 8F shows images of histology of orthotopic tumours derived from transplanted tumour epithelial Aqp5+ and Aqp5− cells. RFP immunohistochemistry highlights the location of the transplanted cells within the gastric mucosa. Immunofluorescence for Aqp5, Muc5ac, Ki67, and Chga indicates that the Aqp5+ cell-derived tumours maintain stem and differentiated lineage marker expression. FIG. 8G shows a schematic of the Aqp5-2A-CreERT2; Apc^{fl/fl}, Pten^{fl/fl}, Trp53^{R172H/+}, Rosa26-tdTomato^{LSL} conditional genetic mouse model for pyloric cancer. FIG. 8H shows representative whole mount and immunostaining images confirming p53 mutant expression (centre) and the presence of Aqp5+ cells in a subset of Aqp5-Cre/APT tumours (right). FIG. 8I shows the FACS gating strategy for isolating tumour epithelial Aqp5+ and Aqp5− cells from Aqp5-Cre/APT tumours. FIG. 8J shows a column graph depicting the relative Aqp5 expression in sorted tumour cell populations by qPCR. FIG. 8K shows images indicating that a larger number of organoids can be established from single Aqp5+ cells compared to Aqp5− cells. FIG. 8L shows a bar graph indicating organoid outgrowth efficiency of single mouse Aqp5+ and Aqp5− cells at P0. Scale bars, 100 μm (a), 500 μm (k), and 1 mm (b, d, h). Graphs represent mean±S.D. with two-tailed unpaired t-test.

FIG. 9 shows results indicating that human Aqp5+ cells function as cancer stem cells. FIG. 9A shows representative brightfield and immunofluorescence images of human pyloric tumour organoids generated from whole glands showing comparable organoid morphology and expression of gastric lineage markers as tumour organoids formed from isolated Aqp5+ tumour cells. FIG. 9B shows a schematic of the Aqp5-2A-CreERT2; AAVS1-CAG-tdTomato^{LSL} constructs inserted into human gastric cancer organoids (top). Genomic PCR performed at the insertion site of Aqp5-2A-CreERT2 confirms successful insertion based on expected band size (bottom). FIG. 9C show brightfield and fluorescent images showing results of a 16 hour 4-OHT induction performed in Aqp5-2A-CreERT2; AAVS1-CAG-tdTomato^{LSL} edited organoids results in the visible tdTomato labelling of a small pool of cells by day 2 that expands to encompass a larger organoid area by day 10. FIG. 9D shows a bar graph quantification of the expansion of tdTomato+ labelled clone area within each organoid over time. FIG. 9E shows brightfield and fluorescent images showing that uninduced Aqp5-2A-CreERT2; AAVS1-CAG-tdTomato^{LSL} organoids never show any visible signs of tdTomato tracing over the same timepoints. FIG. 9F shows imagens of Coimmunofluorescence confirming the overlap of tdTomato-labelled cells and differentiated lineage markers including MUC5AC and KI67. Scale bars, 100 μm. Graphs represent mean±S.D. with two-tailed unpaired t-test.

FIG. 10 shows results indicating that ablation of mouse Aqp5+ cells within a range of pyloric tumour models confirms their requirement for tumour initiation and progression. FIG. 10A shows a schematic of the Aqp5-eGFP-ires-CreERT2; Apd^{fl/fl}; Pten^{fl/fl}, Kras^{LSL-G12D/+}, Rosa26-tdTomato^{LSL} conditional genetic mouse model for pyloric cancer with the addition of a Aqp5-2A-DTR cassette to facilitate diphtheria toxin (DT)-mediated ablation of Aqp5+ cells. FIG. 10B shows representative images showing that Aqp5-Cre/APK and Aqp5-Cre/APK/DTR tumours are comparable in terms of histological features and Aqp5 expression. FIG. 10C shows results indicating that intraperitoneal administration of DT to mice 10 weeks after Tamoxifen-induced pyloric tumour formation results in a ~50% reduction in tumour volume. Representative whole mount images of control and DT-treated tumours are shown on the left. FIG. 10D shows a timeline of DT administration and images of analysis of tumour tissues 12 hours following ablation in Aqp5-Cre/APK/DTR mice (top). Representative immunohistochemistry images confirm the loss of AQP5+ cells in DT-treated Aqp5-Cre/APK/DTR tumours accompanied by an increase in CAS3 and KI67 expression in the remaining tumour load. FIG. 10E shows a timeline of DT administration and analysis of tumour tissues 12 hours following ablation in Aqp5-Cre/APK mice lacking the Aqp5-2A-DTR allele (top). Representative immunohistochemistry images show that control and DT-treated tumours are comparable in terms of AQP5, CAS3 and KI67 levels. FIG. 10F shows data indicating that mouse pyloric tumour organoids generated from Aqp5-Cre/APK/DTR tumours recapitulate AQP5 expression in a subset of cells. Coimmunofluorescence of AQP5 and ECAD in a representative tumour organoid is shown (top). FACS analysis shows that 9-10% of cells within Aqp5-Cre/APK/DTR organoids are highly expressing AQP5 (bottom). A mouse pyloric tumour organoid line lacking AQP5-GFP was used as a control to set the FACS gates in the profile. FIG. 10G shows results indicating that DT treatment of Aqp5-Cre/APK/DTR organoids results in an 80% and 95% decrease in Aqp5 levels relative to control untreated organoids during the organoid outgrowth and organoid expansion phases respectively. FIG. 10H shows data indicating that DT treatment of Aqp5-Cre/APK organoids lacking the Aqp5-2A-DTR allele on day 0 does not affect organoid outgrowth. FIG. 10I shows results indicating that DT treatment of Aqp5-Cre/APK organoids lacking the Aqp5-2A-DTR allele on day 3 does not affect subsequent organoid maintenance. FIG. 10J shows data indicating that DT treatment of Aqp5-Cre/APK organoids lacking the Aqp5-2A-DTR allele does not affect Aqp5 levels at both the organoid outgrowth and organoid expansion phases by qPCR. FIG. 10K to FIG. 10N show timelines highlighting the administration of DT to mice following organoid transplantation for ablation of Aqp5+ cells in vivo and the point at which tissues were harvested for analysis (top). Representative H&E and AQP5 immunohistochemistry images confirm that AQP5 expression was lost and/or perturbed 1 day following ablation either 3 days (FIG. 10K) or 4 weeks (FIG. 10L) after transplantation of Aqp5-Cre/APK/DTR organoids. Representative H&E and AQP5 immunohistochemistry images confirm that DT administration did not affect tumour load or AQP5 expression 4 weeks (FIG. 10M) or 8 weeks (FIG. 10N) following transplantation of Aqp5-Cre/APK organoids lacking the Aqp5-2A-DTR allele. Scale bars, 500 μm (FIG. 10F, FIG. 10H, FIG. 10I) and 1 mm (FIG. 10B, FIG. 10D, FIG. 10E, FIG. 10K, FIG. 10L, FIG. 10M, FIG. 10N). Graphs represent mean±S.D. with two-tailed unpaired t-test.

FIG. 11 shows results indicating that ablation of human Aqp5+ cells within human gastric cancer organoid models confirms their requirement for tumour progression. FIG. 11A shows an image of an agarose gel. Genomic PCR performed at the insertion site of Aqp5-2A-iCaspase confirms successful insertion based on expected band size. FIG. 11 shows immunofluorescence images of AQP5, KI67, MUC5AC, and CHGA confirming the presence of expected lineage markers within Aqp5-2A-iCaspase organoids. FIG. 11C shows the results of dimerizer treatment of 0116 Aqp5-2A-iCaspase organoids resulting in a ~60% decrease in Aqp5 levels relative to control untreated organoids. FIG. 11D shows the results of dimerizer treatment of unedited 0116 parental organoids, which was shown not to alter organoid initiation or subsequent growth tracked over 10 days in culture. FIG. 11E shows images showing that ablation of Aqp5+ cells in an independent GC10 Aqp5-2A-iCaspase organoid line recapitulates the reduced growth rates. FIG. 11F shows a bar graph indicating that dimerizer treatment of GC10 Aqp5-2A-iCaspase organoids results in a 70% decrease in Aqp5 levels relative to control untreated organoids. FIG. 11G is a bar graph indicating that dimerizer treatment of unedited GC10 parental organoids does not alter organoid initiation or subsequent growth tracked over 10 days in culture. FIG. 11H shows a bar graph showing that Aqp5 levels remain unchanged in control and dimerizer-treated GC10 parental organoids by qPCR. Scale bars, 100 μm (FIG. 11B) and 500 μm (FIG. 11D, FIG. 11E, FIG. 11G). Graphs represent mean±S.D. with two-tailed unpaired t-test.

FIG. 12 shows images indicating that targeted ablation of Aqp5+ tumour cells severely impairs organoid and tumour growth. FIG. 12A shows schematics and images depicting the results of diphtheria toxin (DT)-mediated targeted ablation of Aqp5+ tumour cells within pyloric tumour organoids harbouring an Aqp5-2A-DTR allele. In this model, Aqp5− expressing tumour cells concurrently express the DT receptor, rendering them sensitive to DT administration. Addition of DT to ablate Aqp5+ tumour cells in organoids at the time of seeding (Day 0) or in the middle of the organoid growth phase (Day 3) both result in complete lethality of tumour organoids. FIG. 12B shows schematics and images of the results of DT ablation of Aqp5+ tumour cells within pyloric tumour organoids transplanted into the gastric submucosa of immune-deficient NOG mice. Ablation performed at point of tumour initiation (Day 3) completely abolishes tumour formation in DT-treated mice. When ablation is performed at later stages of tumour growth (Week 4), DT-treated tumours display milder tumourigenic phenotypes.

FIG. 13 shows images indicating minimal Aqp5 expression within most of the major human tissues. FIG. 13A shows selected tissue microarray images from brain, heart, kidney, and liver showing absence of or weak Aqp5 staining. In contrast, Aqp5 is strongly expressed at the membrane in salivary glands and testes. FIG. 13B shows images and results of scoring of 47 normal human lung tissue microarray cores, with the vast majority of samples displaying either absence of Aqp5 expression or non-membranous localization of Aqp5 protein.

FIG. 14 shows images indicating that Aqp5 is upregulated in mouse and human gastric tumours. Immunohistochemistry of Aqp5 on human (top) and mouse (bottom) gastric tissues. Aqp5 expression is restricted to gland bases in human and mouse pyloric glands but is extensively and highly expressed within both intestinal- and diffuse-type gastric tumours.

FIG. 15 shows images of analysis of a previously unknown human gastric cancer organoid line. The cell line has been integrated with a Flip-Puro system enabling conditional knockout of Aqp5 upon administration of Cre recombinase gesicles. Loss of Aqp5 in these organoids was confirmed by western blotting. Following Aqp5 knockout in the organoid cells, a reduced cell viability of organoids was shown.

FIG. 16 shows data indicating that Aqp5 drives gastric tumourigenesis. FIG. 16A shows images of Aqp5 wildtype (WT) and knockout (KO) mouse pyloric tumour histology. Representative whole mount (left) and H&E (middle) images are shown, with quantification of tumour volumes (right). FIG. 16B shows significant gene ontology (GO) terms represented in DEGs enriched in Aqp5 WT cancer stem cells (top) and in Aqp5 KO cancer stem cells (bottom). FIG. 16C shows data indicating that human gastric cancer organoids (parental) and four independent Aqp5 KO clones derived from the parental line (clones A1, A5, C2, and C5). AQP5 is expressed in apical membranes of the parental organoids but absent from the KO clones (top). Reduced EdU staining is detected within Aqp5 KO organoids compared to parental (bottom). FIG. 16D shows quantification of cell proliferation within parental and Aqp5 KO organoids using an in vitro MTS colorimetric assay (left) and EdU staining assay (right). FIG. 16Eshows representative whole mount (top) and H&E (bottom) images of orthotopic tumours derived from transplanted parental and Aqp5 KO human gastric cancer organoids. FIG. 16F shows results of quantification of tumour volumes (left) and invasion rate (right) of orthotopic tumours derived from transplanted parental and Aqp5 KO human gastric cancer organoids. FIG. 16G shows a schematic of the FLIP-Puro cassette used in generating Cre-inducible Aqp5 KO in human gastric cancer organoids. FIG. 16H shows images indicating the conditional KO of Aqp5 by Cre recombinase treatment in human gastric cancer organoids reduces organoid growth in vitro. FIG. 16I shows the results of quantification of relative organoid numbers at the 5-day timepoint following conditional Aqp5 KO. Organoids at least 100 μm in diameter were included in the analysis. FIG. 16J shows a schematic of significant GO terms enriched in untreated and Cre-induced Aqp5 KO organoids at various indicated timepoints post-induction. Scale bars, 100 μm (FIG. 16C-bottom), 500 μm (FIG. 16C-top, FIG. 16H) and 1 mm (FIG. 16A, FIG. 16E-bottom). Graphs represent mean±S.D. with two-tailed unpaired t-test.

FIG. 17 shows results indicating that AQP5 drives tumour progression in multiple human gastric cancer cell lines. FIG. 17A shows results of the analysis of AQP5 functions in the intestinal-type AGS gastric cancer cell line. Representative immunofluorescence images and qPCR of Aqp5 levels (left) confirm the upregulation of Aqp5 in three AQP5-overexpressing (OE) clones. In vitro assays show that OE clones have higher cell proliferation and cell migration rates in culture (right). FIG. 17B shows the analysis of AQP5 functions in the diffuse-type SNU601 gastric cancer cell line. Representative immunofluorescence images and qPCR of Aqp5 levels (left) confirm the upregulation of Aqp5 in three AQP5-overexpressing (OE) clones. In vitro assays show that OE clones have higher cell migration rates in culture (right). FIG. 17C shows results indicating that AGS OE cells establish orthotopic tumours in mice that are larger and display more aggressive tumour features. FIG. 17D shows the analysis of AQP5 functions in the diffuse-type KATOIII cell line expressing high levels of AQP5. Representative immunofluorescence images and qPCR of Aqp5 levels confirm the loss of Aqp5 in knockout (KO) cells (left). In vitro assays show that KO cells proliferative more slowly in culture compared to the parental cells (right). FIG. 17E shows that KATOIII KO cells fail to establish orthotopic tumours in mice in the majority of cases examined. Scale bars, 500 μm (FIG. 17A, FIG. 17B, FIG. 17D) and 1 mm (FIG. 17C, FIG. 17E). Graphs represent mean±S.D. with two-tailed unpaired t-test.

FIG. 18 shows data indicating that AQP5 knockout in a pyloric cancer mouse model represses tumour growth and displays an altered transcriptomic profile. FIG. 18A shows representative immunofluorescence images of Aqp5 WT and Aqp5 KO pyloric tumours confirming the loss of AQP5 in the KO tumours. FIG. 18B shows data indicating that mouse pyloric tumour organoids generated from Aqp5 WT and Aqp5 KO tumours reflect the pattern of AQP5 expression in the original tumours. FIG. 18C shows that Aqp5 KO tumour organoids form less invasive tumours following transplantation into immunodeficient mice compared to Aqp5 WT tumour organoids. Representative whole mount and H&E images are shown (left), and quantifications of tumour volume and invasion rates (right). FIG. 18D shows the FACS gating strategy for isolation of tumour epithelial GFP+ (stem) and GFP− (rest of tumour) cells from Aqp5 WT and Aqp5 KO mouse pyloric tumours. FIG. 18E shows a heatmap of the top 50 differentially expressed genes (DEGs) between GFP+ and GFP− samples isolated from 3 independent mouse pyloric tumours. FIG. 18F shows a Venn diagram of the DEGs common and unique between the Aqp5 KO and Aqp5 WT tumour profiles. FIG. 18G shows the results of reactome analysis for genes enriched in Aqp5 WT tumours highlighting potential pathways downstream of Aqp5 functions. Scale bars, 500 μm (FIG. 18B) and 1 mm (FIG. 18A, FIG. 18C). Graphs represent mean±S.D. with two-tailed unpaired t-test.

FIG. 19 shows data indicating that AQP5 drives tumour progression in multiple human gastric cancer organoid lines. FIG. 19A, FIG. 19B, and FIG. 19C show results obtained in three independent human gastric cancer organoid lines 0045 (FIG. 19A), 0235 (FIG. 19B), and 0116 (FIG. 19C) used for analysis of AQP5 functions in vitro. Representative immunofluorescence images (left) and qPCR of Aqp5 levels (middle) confirm the upregulation of Aqp5 in AQP5-overexpressing (OE) organoid lines. Of note, Aqp5 upregulation in 0116 OE organoids is more modest compared to the other two OE lines as Aqp5 is already strongly expressed in the parental 0116 line. The 0045 OE and 0235 OE organoid lines show greater cell proliferation rates in vitro compared to their respective parental lines as measured by an MTS proliferation assay (right). FIG. 19D, FIG. 19E, and FIG. 19F show results of orthotopic transplantation of the three human gastric cancer organoid lines 0045 (FIG. 19D), 0235 (FIG. 19E), and 0116 (FIG. 19F) used for analysis of AQP5 functions in vivo. Representative whole mount and H&E images (left) depict the histological features of the tumours formed from transplanted organoids. Quantifications of tumour volume (middle) and invasion rate (right) show that OE organoids are capable of forming large tumours with more invasive features compared to their respective parental lines. FIG. 19G shows the results of genomic PCR performed at the insertion site of Aqp5-FLIP-Puro confirms successful insertion based on expected band size. FIG. 19H shows data indicating that AQP5 levels are significantly reduced in Aqp5 FLIP-Puro organoids following Cre recombinase treatment. Scale bars, 500 μm (FIG. 19A, FIG. 19B, FIG. 19C) and 1 mm (FIG. 19D, FIG. 19E, FIG. 19F). Graphs represent mean±S.D. with two-tailed unpaired t-test.

### DETAILED DESCRIPTION

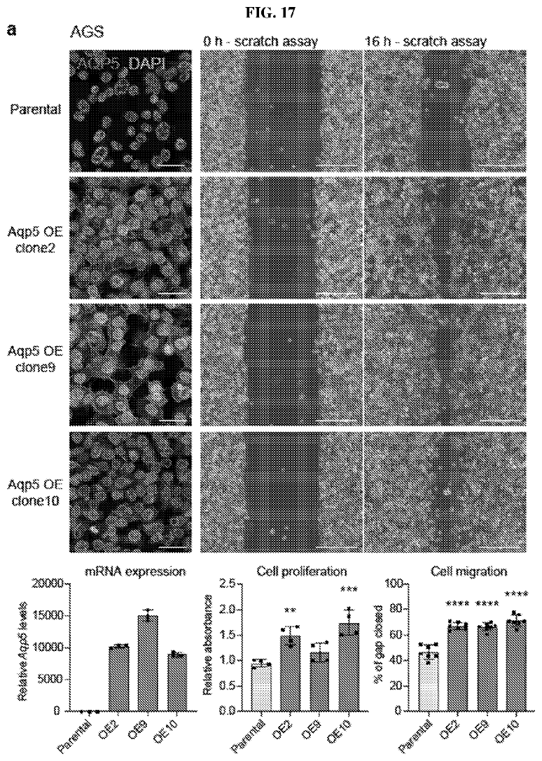

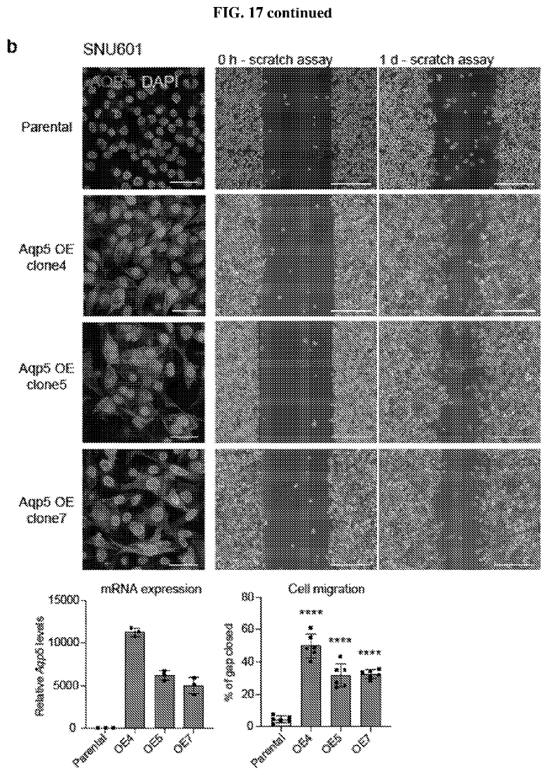

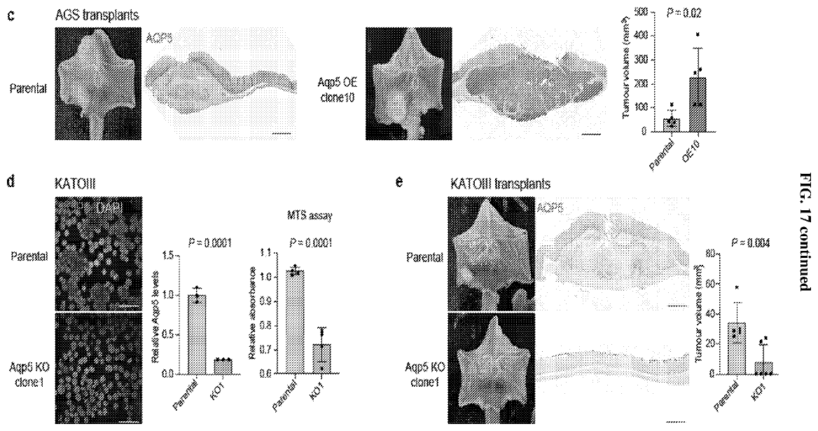

While Aqp5 has been implicated in driving tumour cell proliferation and migration in human gastric cancer cell lines, its precise mechanism of action remains unclear. In gastric tumours, Aqp5 is frequently expressed at elevated levels, but the role of Aqp5 in gastric cancer has not been thoroughly explored in near-physiological cancer organoid and mouse models. The human gastric cancer cell line AGS, which is derived from an intestinal-type tumour, presents modest levels of Aqp5 in vitro (FIG. 17A). Three independent Aqp5-overexpressing AGS cell lines were generated with highly elevated Aqp5 levels and found that they presented significantly increased cell proliferation and cell migration rates in vitro (FIG. 17A), in agreement with a previous study. In a separate diffuse-type gastric cancer cell line, SNU601, elevated cell migration rates were detected in vitro in three independent Aqp5-overexpressing lines (FIG. 17B). Moreover, following orthotopic transplantation into the pylorus of immune-deficient mice, Aqp5-overexpressing AGS cells formed larger tumours that invaded aggressively into the epithelial and muscle layers (FIG. 17C). Conversely, knocking out Aqp5 in the gastric cancer cell line KATOIII, which expresses high levels of Aqp5, resulted in reduced cell proliferation rates in vitro (FIG. 17D). While parental KATOIII cells seeded large tumours in the gastric submucosa following orthotopic transplantation, Aqp5-null KATOIII cells failed to establish tumours in the majority of mice analysed (FIG. 17E), indicating that Aqp5 could contribute to a critical step in tumour establishment. Thus, the data disclosed herein shows that Aqp5 functions in promoting tumourigenic traits of cell proliferation and migration in gastric cancer cell lines and further extends these findings to an in vivo context through transplantation assays.

Described in the present disclosure is the identification of Aqp5 as marker of gastric cancer stem cells, which has been identified using multiple assays across physiologically-relevant mouse and human gastric cancer models. The Aqp5 surface marker is shown herein to facilitate efficient isolation of prospective gastric cancer stem cells for expression profiling, to identify additional markers and potential therapeutic vulnerabilities. The elimination of the Aqp5-expressing tumour cell population as shown herein is an example of a therapeutic approach used to ameliorate gastric cancer progression.

### Aqp5 is a Functional Gastric Cancer Stem Cell Marker Driving Tumourigenesis

Cancer stem cells make up a self-renewing population capable of generating differentiated tumour cell lineages and fueling tumour growth. In gastric tumours, markers of cancer stem cells have been proposed, but to date these have largely remained restricted to animal cancer models, are broadly expressed in many normal and cancerous tissues, or have failed to be robustly validated by functional assays demonstrating cancer stem cell potential in near-physiological mouse and human gastric cancer models. Crucially, the identification of such markers facilitates the isolation of cancer stem cells to decipher downstream mechanistic functions and the development of targeted therapies against this cell population in human gastric tumours, which could support a longer-term remission of the disease. In normal tissues, Aqp5 marks mouse and human pyloric stem cells, but dysregulation of major pathways altered in gastric cancer in these Aqp5+ cells is sufficient to drive the formation of pyloric tumours in mouse models. Here, using multiple, representative in vitro and in vivo gastric cancer models, Aqp5 is shown to be a functionally validated gastric cancer stem cell marker present in both mouse and human gastric tumours. This shows the requirement and contribution of Aqp5+ tumour cells in sustaining tumour growth. These findings improve the understanding of the cancer stem cell model, enabling the development of diagnostic tools and therapeutic strategies targeting gastric cancer.

Thus, the present disclosure describes the use of Aqp5 as a marker expressed by a gastric cancer stem cell pool. In one example, the marker disclosed herein is used to facilitate selective targeting and/or elimination of a gastric cancer stem cell population, resulting in an effect on disease progression. In a further example, Aqp5 is used as a marker to identify a gastric cancer. In another example, there is disclosed a biomarker of gastric cancer stem cells, wherein the biomarker is AQP5. In another example, the biomarker is membrane-bound AQP5.

In a further example, a method of identifying a gastric cancer stem cell or gastric cancer is disclosed. In one example, the method disclosed herein comprises a1) detecting the expression of Aquaporin 5 (AQP5) in a cell or cell population; or b1) detecting the expression level of AQP5 in a cell or cell population and comparing the expression level with the expression level of AQP5 in a reference cell or reference cell population, wherein detection of AQP5 expression in the cell or cell population, or expression of AQP5 in the cell or cell population at an increased level compared to the reference cell or reference cell population identifies said cell or cell population as a gastric cancer stem cell or gastric cancer stem cell population.

In another example, the method disclosed herein further comprise isolating the identified gastric cancer stem cell or stem cell population.

In one example, the cell or cell population disclosed herein is an in vitro, in vivo, ex vivo cell or cell population. In another example, the method disclosed herein is a method that is performed in vitro, in vivo or ex vivo.

In yet another example, the cell or cell population disclosed herein is a gastric tumour sample, biopsy, or organoid. Examples of a cell as disclosed herein can be, but are not limited to, an epithelial cell. In another example, the cell population disclosed herein comprises epithelial cells from the gastric tumour sample or biopsy.

In one example, the method disclosed herein comprises comparing the expression obtained level with an expression level obtained from a reference cell or cell population. Such a reference cell or cell population can be a cell that does not express AQP5, optionally wherein the cell that does not express AQP5 is a non-gastric cell, a non-cancerous gastric cell, or combinations thereof. In one example, the reference cell or cell population is one that comprises or consists of cells that are non-cancerous gastric cells.

Aqp5 is a marker of gastric cancer stem cells in both mouse and human gastric tumours. Using established protocols to isolate Aqp5+ and Aqp5− cells from gastric tumours, Aqp5+ tumour cells were shown to function as stem cells capable of seeding new tumours and repopulating the tumour bulk when transplanted into mouse recipients. Moreover, isolated Aqp5+ tumour cells selectively form organoids in vitro that can be maintained in long-term culture. These findings were obtained using a comprehensive range of mouse and human gastric cancer models developed in-house. These assays collectively demonstrate the cancer stem cell potential presented by the Aqp5+ tumour cell population and identify Aqp5 as a gastric cancer stem cell markers validated in near-physiological models of human gastric cancer.

Aqp5 Marks a Subset of Epithelial Cells within Mouse and Human Pyloric Tumours

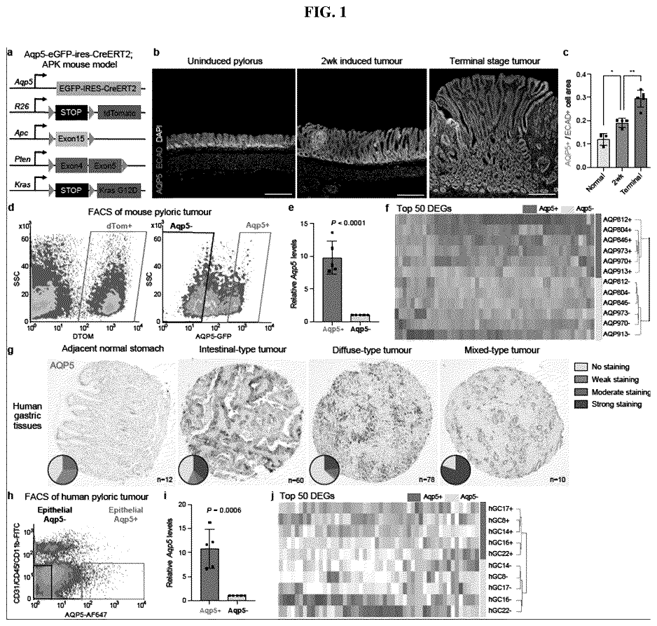

To study the contribution of Aqp5+ tumour cells towards gastric cancer, the expression of Aqp5 was first characterised within mouse pyloric tumours. Recombination of conditional Apc, Pten, and Kras^{G12D} floxed alleles under an Aqp5-eGFP-IRES-creERT2 driver to recapitulate the major co-dysregulated pathways prevalent in human gastric tumours resulted in the formation of Aqp5-Cre/APK pyloric tumours in these mice within 2 to 3 months classified as tubular-type adenocarcinoma (FIG. 1A, FIG. 4A), as previously described.

Thus, in one example, the cell or cell population disclosed herein is further modified to express AQP5 in conjunction with an inducible gene. In one example, the inducible gene is CreERT2. In another example, the inducible gene is an inducible Caspase9 (iCasp9) gene.

Aqp5 expression was restricted to the tissue-resident stem cell compartment at pyloric gland bases prior to cancer induction, overlapping with Lgr5 (FIG. 1B, FIG. 4C). At later stages of cancer progression, increasing numbers of Aqp5+ tumour cells were detected in the transformed pyloric epithelium, including regions above the gland bases (FIG. 1B, FIG. 1C). As the Aqp5-eGFP-IRES-creERT2 cassette disrupts endogenous expression of Aqp5, spatiotemporal patterns of Aqp5 expression during tumour progression were observed using an alternative Aqp5-2A-creERT2 pyloric cancer model in which endogenous Aqp5 expression is not perturbed (FIG. 4C). In agreement with these histological analyses, single cell RNA sequencing (scRNAseq) of healthy pylorus and Aqp5-Cre/APK pyloric tumours indicated that Aqp5 was elevated in a subset of tumour epithelial cell lineages (data not shown). While Aqp5 and Lgr5 expression was highly restricted to the same stem cell cluster in the healthy pylorus, Aqp5 was shown to be enriched in a largely distinct cellular compartment from Lgr5 within pyloric tumours, with a smaller subset co-expressing the two markers (data not shown).

To situate these Aqp5+ cell subsets within the intact pyloric tumour, imaging-based spatial transcriptomics using MERSCOPE and constructed a spatial cellular map of the mouse Aqp5-Cre/APK tumour was also performed (FIG. 4D). This approach revealed eight (8) clusters corresponding to tumour epithelial lineages (FIG. 4E), three (3) of which showed elevated Aqp5 transcript levels and mapped to both the gland bases and tumour compartments immediately above the gland bases (FIG. 4D, FIG. 4F). Aqp5+ tumour lineages comprised of Wnt-active clusters overlapping with Lgr5+ cells as well as highly proliferative clusters expressing Mki67 (FIG. 4F), in line with the previously obtained scRNAseq dataset. Aqp5 expression was further validated in these mouse pyloric tumours relative to other putative gastric cancer stem cell markers Lgr5, Cxcr4, and Cd44 by RNAscope (FIG. 4G), thereby showing that Aqp5 labels a subpopulation of pyloric tumour cells that has not previously been evaluated.

In one example, the method is as disclosed herein, wherein the method comprises further analysing the isolated gastric cancer stem cells, the stem cell population, or the AQP5-expressing tumour organoid. Methods of analysing the isolated gastric cancer stem cell or the AQP5-expressing tumour organoid can include, but are not limited to, omics analysis such as transcriptomic analysis (for example, RNA sequencing, single-cell RNA sequencing, spatial transcriptomics, gene ontology analysis, polymerase chain reaction analysis or combinations thereof), proteomic analysis (for example, single-cell proteomics) and combinations thereof. In one example, the further analysis is spatial transcriptomics.

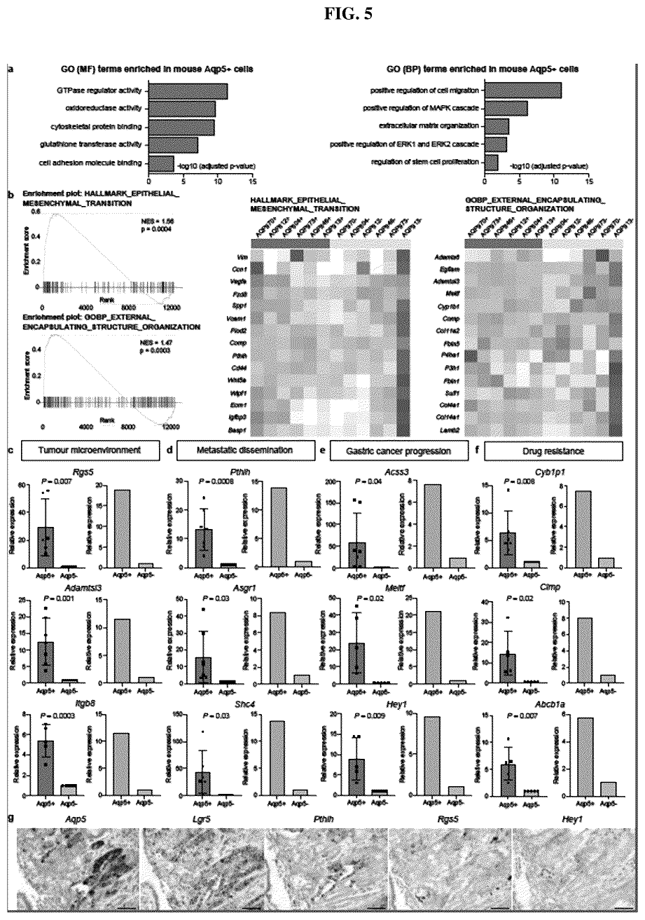

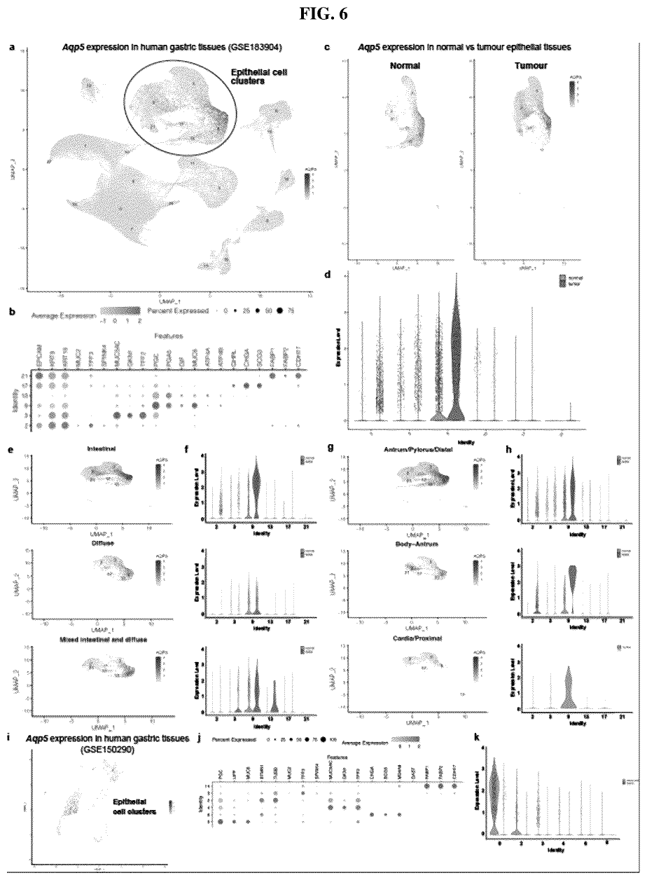

A strategy was established to isolate these pyloric tumour epithelial Aqp5+ and Aqp5− cell populations within Aqp5-Cre/APK tumours by fluorescence-activated cell sorting (FACS) to mine biological insights from Aqp5+ tumour cells (FIG. 1D). The Aqp5-Cre/APK model harbours a Rosa26-tdTomato^{LSL} reporter that robustly traces entire pyloric glands within one week following induction, facilitating the use of tdTomato as a marker to isolate the tumour epithelial population, and eGFP as a reporter of Aqp5 expression. Sorted eGFP+ tumour epithelial cells showed an average of 9.8-fold enrichment in Aqp5 levels by qPCR compared to eGFP-cells (FIG. 1E). Using this sorting strategy, transcriptomic profiling of Aqp5+ and Aqp5− tumour epithelial cells isolated from 6 independent Aqp5-Cre/APK mouse pyloric tumours (FIG. 1F) was performed. This revealed the selective enrichment of extracellular matrix (ECM) remodelling, epithelial-mesenchymal transition (EMT), and drug resistance-associated pathways in the Aqp5+ tumour cell transcriptome (FIG. 5A, FIG. 5B, Table 1), features typically associated with cancer stem cells in other systems. Many of these upregulated genes, including Pthlh, Rgs5, and Hey1, were validated using additional mouse pyloric tumour samples by qPCR and RNAscope (FIG. 5C to FIG. 5G, Table 2), showing their robust enrichment in the Aqp5+ tumour cell population.

Thus, in one example, the cell or cell population disclosed herein is modified to express AQP5 in conjunction with one or more detectable labels. Such detectable labels can be, but are not limited to, fluorescent labels, tags, proteins, and combinations thereof. Non-exhaustive examples of fluorescent labels are tdTomato, GFP, eGFP. RFP, YFP, and combinations thereof. In one example, the fluorescent label is eGFP.

In a further example, the gastric cancer stem cell disclosed herein expresses one or more markers of gastric cancer progression (such as, but not limited to, Pthlh, Hey1, Rgs5), cancer stem cell functions (such as, but not limited to, Hey1, Clmp, Cyb1p1), tumour microenvironment (examples of which are Rgs5, Adamntsl3, Itgb8) and combinations thereof.

Aqp5-expressing stem cells had been previously identified and characterised in the healthy human pylorus. In order to conduct a detailed evaluation of their tumour equivalents, an analysis of Aqp5 expression using publicly available human scRNAseq datasets of healthy stomach and gastric tumour tissues was performed. A total of six (6) epithelial clusters were identified (FIG. 6A), including lineages enriched in gastric mucous (Muc5ac+, Muc6+), chief cell (Pgc+), and enteroendocrine (Chga+) markers, and intestinal (Tff3+, Fabp1+) markers (FIG. 6B). Within the healthy human pylorus, Aqp5 is primarily expressed in the Muc6+ mucous gland base cell cluster, previously associated with the gastric stem cell compartment in both mice and humans (FIG. 6C). This is in contrast to Aqp5 expression in human gastric tumours, which was shown to be increased across multiple epithelial cell clusters (FIG. 6C, FIG. 6D). Aqp5 expression was also seen to display tumour subtype-specific patterns, with the largest elevation in Aqp5 levels detected in intestinal-type tumours, while diffuse-type tumours did not exhibit significant differences in Aqp5 levels compared to healthy tissue (FIG. 6E to FIG. 6H). These results were substantiated using an independent human scRNAseq dataset, where Aqp5 was also significantly elevated in gastric tumour cells over their healthy equivalents (FIG. 6I). This was further substantiated and by detecting Aqp5 at the protein level using IHC performed on human gastric tissue microarrays (FIG. 1G), corroborating with previous work. Collectively, these analyses identify an elevated Aqp5 expression signature in human gastric tumours primarily associated with intestinal-type tumours, features that are robustly captured in the Aqp5-Cre/APK mouse pyloric cancer model disclosed herein.

Thus, in one example, the method disclosed herein comprises detecting the expression level of AQP5. Such an expression level can be detected as, for example, gene expression level, protein expression level, or a combination thereof. In one example, the method is as described herein, wherein the step of detecting the protein expression level of AQP5 is performed. In another example, the method is as described herein, wherein the step of detecting the gene expression level of AQP5 is performed.

In another example, the protein expression level of AQP5 can be detected using methods such as, but not limited to, immunohistochemistry (IHC), flow cytometry, Western blot, and combinations thereof. In one example, the protein expression level of AQP5 is detected using immunohistochemistry. In another example, the expression level of AQP5 is a gene expression level. As such, the gene expression level of AQP5 can be obtained or quantified by, for example, performing a polymerase chain reaction (PCR).

As Aqp5 is a membrane-bound protein, the sorting protocols performed herein were adapted to perform antibody-based FACS isolation of Aqp5+ and Aqp5− human pyloric tumour epithelial cells from freshly collected patient tumour biopsies (FIG. 1H).

Thus, the methods disclosed here can also comprises a step of isolating one or more identified gastric cancer stem cells. Methods of isolating such cells include, but are not limited to, single cell sorting, fluorescent activated cell sorting (FACS), magnetic sorting, or combinations thereof. In one example, the cells are isolated using fluorescent activated cell sorting (FACS).

In another example, there is described a method of isolating one or more gastric cancer stem cells from a cell population, comprising i) contacting cells of the cell population with an agent that binds to AQP5; ii) isolating one or more AQP5-expressing cells that are bound to the agent, wherein the one or more AQP5-expressing cells are gastric cancer stem cells.

Examples of such agents that bind to AQP5 can be, but are not limited to, antibodies, drugs, small molecules, and combinations thereof. In one example, the agent that binds to AQP5 is an antibody. In one example, the agent that binds to AQP5 may affect the function of AQP5 once bound. In another example, the antibody is a detection antibody. In another example, the antibody can be conjugated to a label, such as a detectable label. Examples of such detectable labels are, but are not limited to, fluorescent labels, cleavable labels, isolation labels, purification labels, and combinations thereof. In another example, the agent is conjugated to a compound such as a drug, or small molecule.

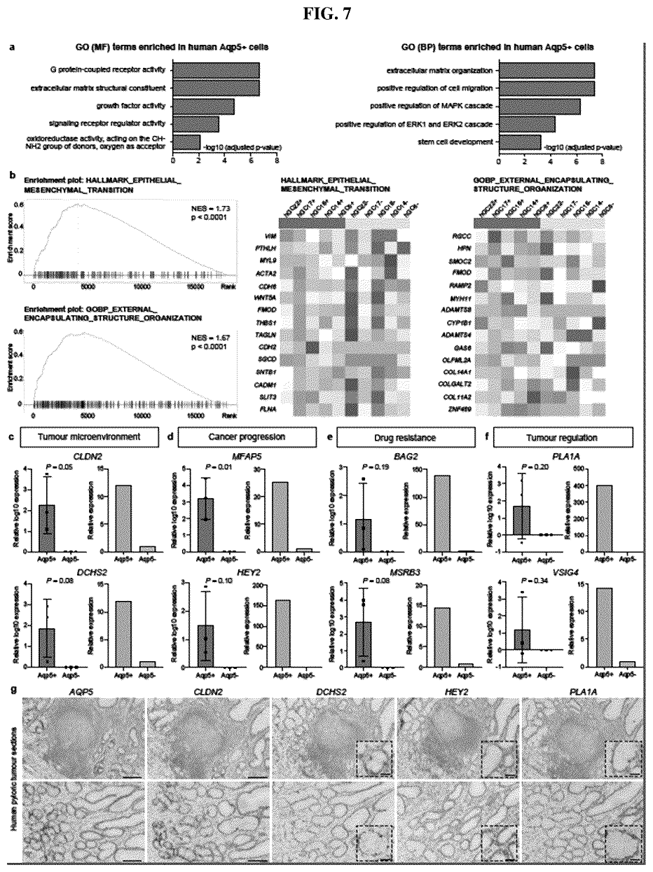

This FACS sorting strategy was validated by qPCR reflecting a mean of 10.8-fold upregulation of Aqp5 levels in the sorted Aqp5+ cell population (FIG. 1I). Using this sorting approach, bulk RNAseq analyses on isolated Aqp5+ and Aqp5-human pyloric tumour cells from five (5) independent tumour samples was performed (FIG. 1J). Like the mouse Aqp5+ profile, human Aqp5+ tumour cells displayed a transcriptomic signature enriched in cancer stem cell-associated phenotypes, including ECM organization, EMT, and chemoresistance (FIG. 7A, FIG. 7B, Table 3). Selected upregulated targets, including CLDN2, HEY2, and PLA1A, were further validated by qPCR and RNAscope using additional independent patient samples (FIG. 7C to FIG. 7G, Table 4), confirming the reliability of the RNAseq dataset. In all, mouse and human Aqp5+ pyloric tumour cells presented common cancer stem cell-enriched transcriptomic profiles that are distinct from the Aqp5+ healthy pyloric stem cell signature, thereby highlighting its therapeutic value.

### Aqp5+ Tumour Cells Function as Cancer Stem Cells in Mouse and Human Pyloric Tumours

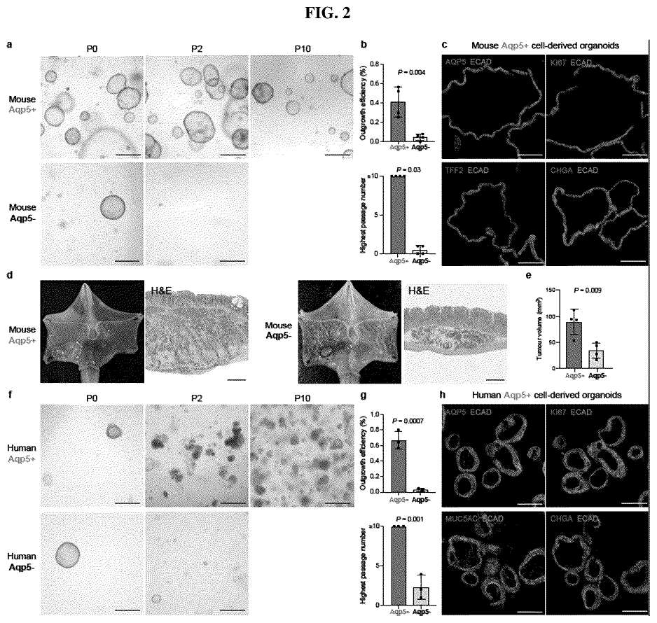

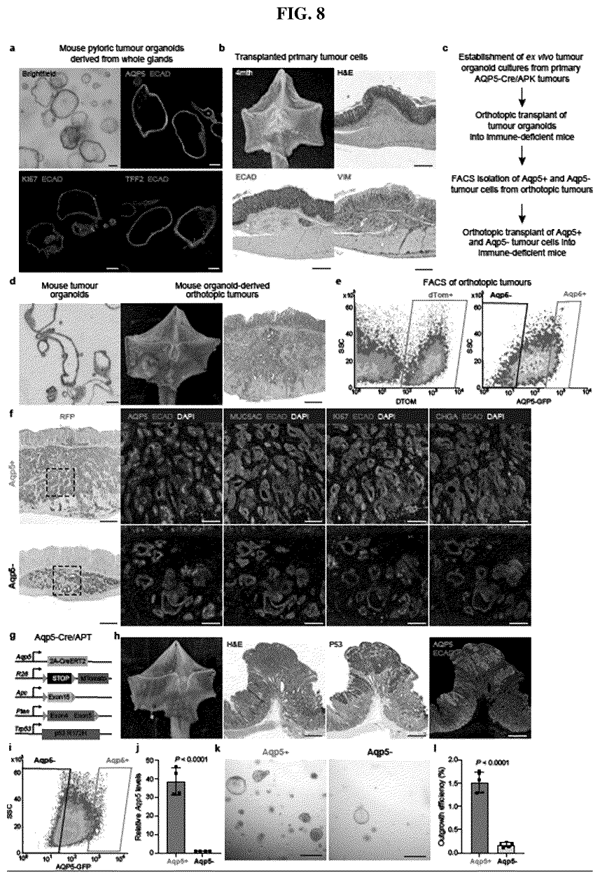

Markers that label healthy and cancer stem cells are known to be highly overlapping in many tumour contexts. However, many of gastric cancer stem cell markers, including Lgr5, Cd44, and Cxcr4, have not been robustly validated using functional assays directly demonstrating the stem potential of their labelled tumour cell population. Given that Aqp5 marks the healthy mouse and human pyloric stem cell compartment, and many reported cancer stem cell-associated pathway signatures are enriched in the transcriptomes derived from Aqp5+ tumour cells (FIG. 5 and FIG. 7), it was asked if Aqp5 could also be a specific marker of the pyloric cancer stem cell population within mouse and human gastric tumours. To functionally test this hypothesis, Aqp5+ and Aqp5− epithelial tumour cells were isolated from Aqp5-APK mouse pyloric tumours by FACS and seeded for organoid culture. This revealed organoid formation by Aqp5+ tumour cells that was maintained long-term (at least 10 passages) (FIG. 2A, FIG. 2B). Moreover, organoids derived from isolated Aqp5+ tumour cells contained multiple differentiated cell lineages that resembled organoids generated from whole pyloric tumour glands (FIG. 2C, FIG. 8A). In contrast, sorted Aqp5-tumour cells formed a significantly smaller number of organoids in culture that were rapidly lost within 2 passages (FIG. 2A, FIG. 2B), reflecting a lack of self-renewal capacity. Thus, Aqp5 was shown to mark a functional cancer stem cell population, within mouse pyloric tumours, capable of long-term tumour organoid growth.

In one example, the methods disclosed herein comprise a step of further culturing the isolated gastric cancer stem cell in the presence of culture media that does not comprise growth factors. In other words, in one example, the step of further culturing the isolated gastric cancer stem cell takes place without the presence of growth factors in the cell culture medium. In another example, the method disclosed herein comprises a step of isolating a gastric cancer stem cell, wherein the isolated gastric stem cell forms an AQP5-expressing tumour organoid.

In one example, the gastric cancer stem cell is a mammalian cell. Examples of such mammalian cells are, but are not limited to, murine (mouse) cells and human cells.

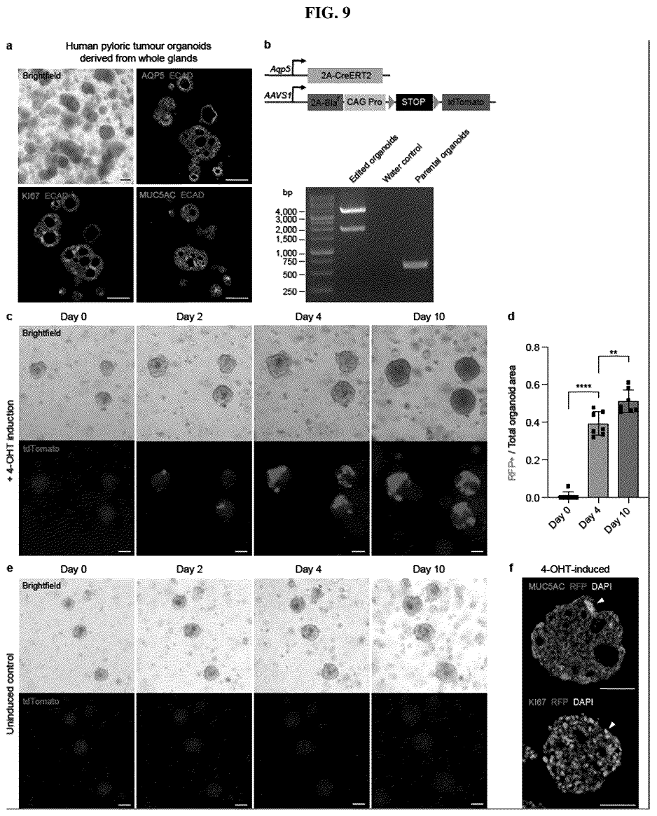

Another approach to assess cancer stem cell potential is by transplantation of specific cell populations into mice to evaluate their tumour-forming capacity in an in vivo context. As known in the art, it remains challenging to transplant sorted cell populations from solid tumours, and efficiency of tumour initiation can vary markedly depending on the tissue type and recipient. In agreement with this observation, tumour cells dissociated from Aqp5-Cre/APK tumours failed to establish sizeable tumours following orthotopic transplantation into the pylorus of immune-deficient mice even after 4 months (FIG. 8B). To circumvent this issue, Aqp5-Cre/APK pyloric tumour organoids into were transplanted into immune deficient mice. FACS sorted Aqp5+ and Aqp5− cells from the orthotopic tumours were then derived from these mice (FIG. 8C to FIG. 8E), as it was thought that these cells would have better adapted to the local tumour environment within their hosts. It was shown that sorted Aqp5+ and Aqp5− tumour epithelial cells from Aqp5-Cre/APK orthotopic tumours seeded new tumours in mice more efficiently than cells isolated from primary tumours, thereby offering a viable system for evaluating their tumour initiation capacities (FIG. 8F). Across the five (5) independent sets of mice examined, Aqp5+ tumour cells were shown to consistently establish orthotopic tumours that were larger in volume and displayed more aggressive proliferation and invasive features, compared to Aqp5− tumour cells derived from the same tumour (FIG. 2D, FIG. 2E, FIG. 8F). Aqp5+ cell-derived tumours were also shown to comprise both Aqp5+ and Aqp5− cells, including the expression of multiple differentiated cell lineage markers (FIG. 8F), indicating that these Aqp5+ cancer stem cells have the capacity to reconstitute the diverse lineages within their original tumours. Finally, Aqp5+ and Aqp5− tumour cells were isolated from a pyloric cancer mouse model incorporating the constitutively active p53^{R172H} mutant (FIG. 8G, FIG. 8H), in order to evaluate the stem potential of Aqp5+ cells in an independent tumour context. FACS isolation of these cells was validated by qPCR, reflecting a 38-fold enrichment of Aqp5 in the sorted Aqp5+ population (FIG. 8I, FIG. 8J). In this cancer model, sorted Aqp5+ tumour cells were also seen to form higher numbers of organoids in vitro compared to Aqp5− cells derived from the same tumour (FIG. 8K, FIG. 8L), indicating that Aqp5+ cells could also function as stem cells in this context. Taken together, the organoid initiation and transplantation assays establish mouse Aqp5+ tumour cells as a functional gastric cancer stem cell population capable of driving tumour growth. To evaluate whether human Aqp5+ tumour cells also function as cancer stem cells, isolated single Aqp5+ and Aqp5− human pyloric tumour cells were isolated from primary tumour samples and plated for organoid culture. It was found that human Aqp5+ tumour cells also generated tumour organoids at a higher rate than the corresponding Aqp5− cells isolated from the same tumour (FIG. 2F, FIG. 2G). The Aqp5+ tumour cell-derived organoids displayed long-term self-renewal capabilities (at least 10 passages) and gave rise to differentiated cell lineages resembling human organoids established from whole pyloric glands (FIG. 2H, FIG. 9A), suggesting that the Aqp5+ cell population retained an intrinsic stem capacity of repopulating the cell lineages and characteristics of its original tumour. In contrast, the small proportion of Aqp5− cell-derived organoids that formed failed to propagate beyond an average of 3 passages (FIG. 2F, FIG. 2G).

As an additional readout of stem cell potential of the human Aqp5+ tumour cell population, human gastric tumour organoids with CRISPR/Cas9-mediated insertion of Aqp5-2A-CreERT2; CAG-LSL-tdTomato, enabling in vitro lineage tracing of human Aqp5+ cells and their progeny within these tumour organoids, were established (FIG. 9B). Administration of 4-OHT to these tumour organoid cultures resulted in the labelling of a small pool of Aqp5+ cells that expanded to encompass an average of 51% of the organoid area after 10 days (FIG. 9C to FIG. 9E), overlapping with differentiated gastric lineage markers (FIG. 9F). In contrast, uninduced organoids were not seen to display tdTomato tracing in vitro across the same timepoints (FIG. 9E). Thus, human Aqp5+ tumour cells from both primary patient samples and human organoid models were shown to function as stem cells that directly contribute to the long-term maintenance of organoid cultures and generation of multiple differentiated cell lineages.

### Ablation of Aqp5+ Tumour Cells Impairs Tumour Development

Cancer stem cells have been attributed as a major source of tumour relapse as they are capable of resisting standard cancer therapies, for example, by activating drug efflux transporters. Methods of targeting cancer stem cells have thus been proposed as a way of eliminating tumour burden and recurrence, but this has been hindered by the lack of well-validated and clinically relevant markers of cancer stem cells. In view of the functional characterisation of mouse and human Aqp5+ cells as a cancer stem cell population in pyloric tumours (FIG. 2), the effect of ablating Aqp5+ cells in mouse and human gastric tumour models were tested as an independent functional evaluation of their cancer stem cell identity. The effectiveness of targeting the Aqp5+ cancer stem cell population as a therapeutic approach against gastric cancer was also evaluated.

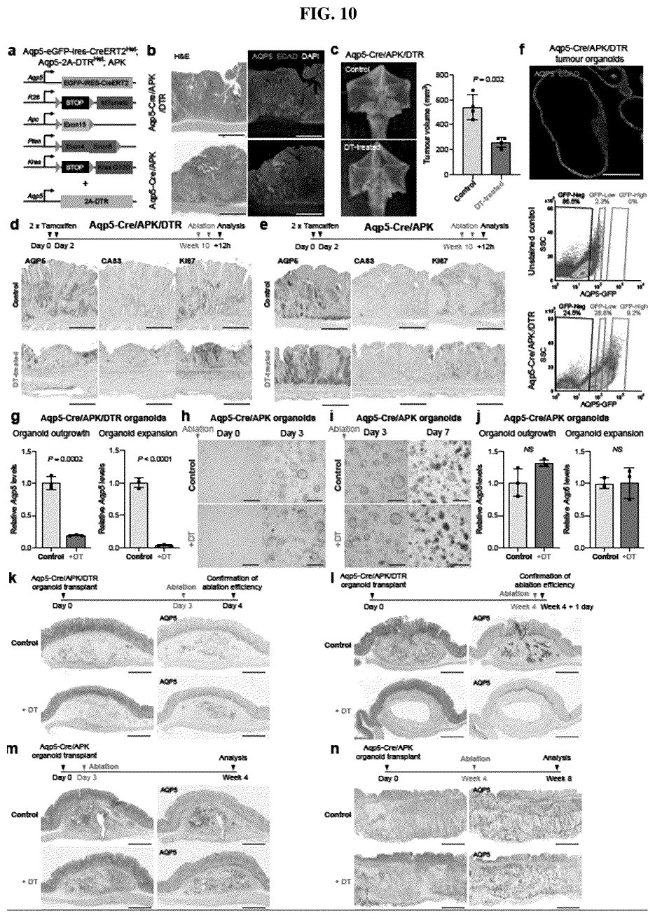

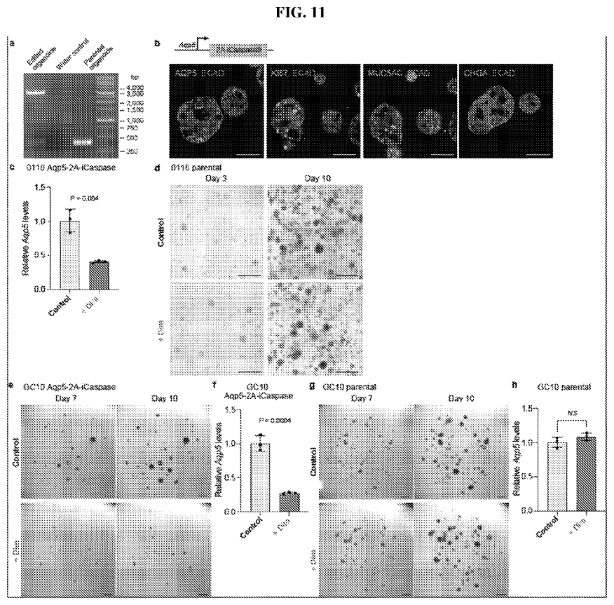

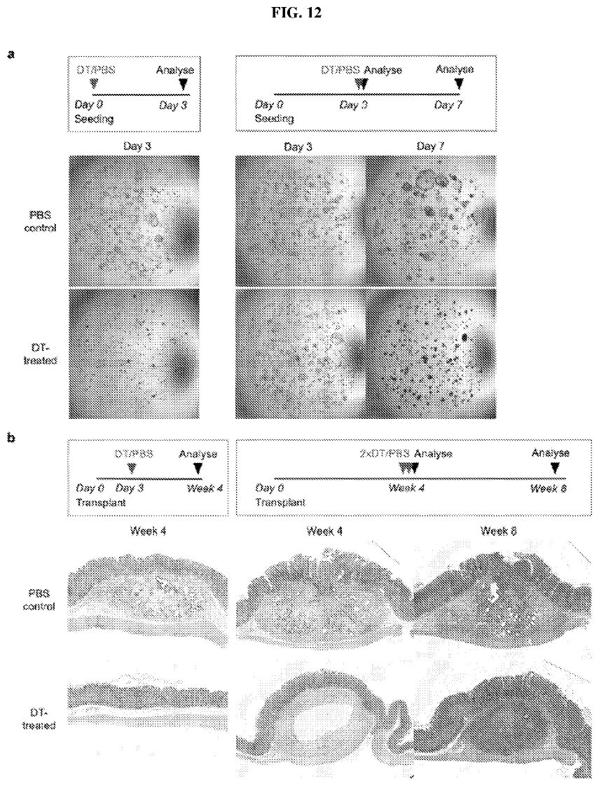

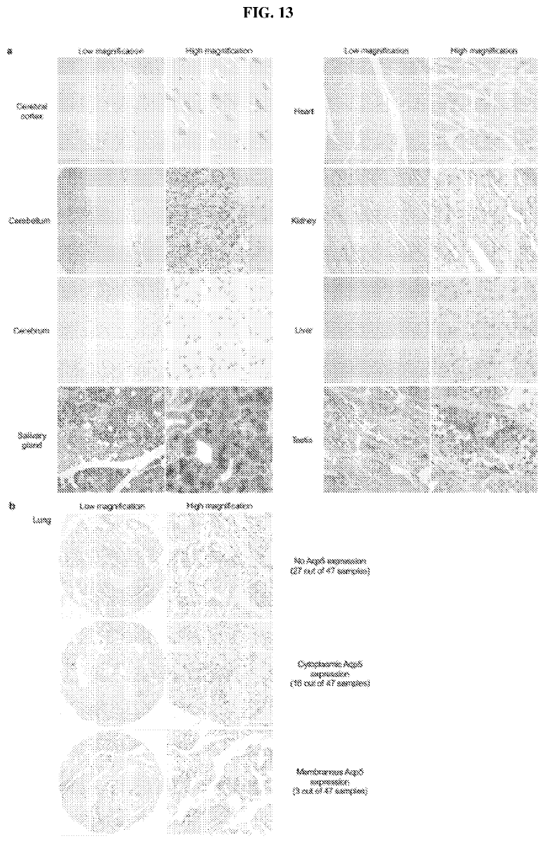

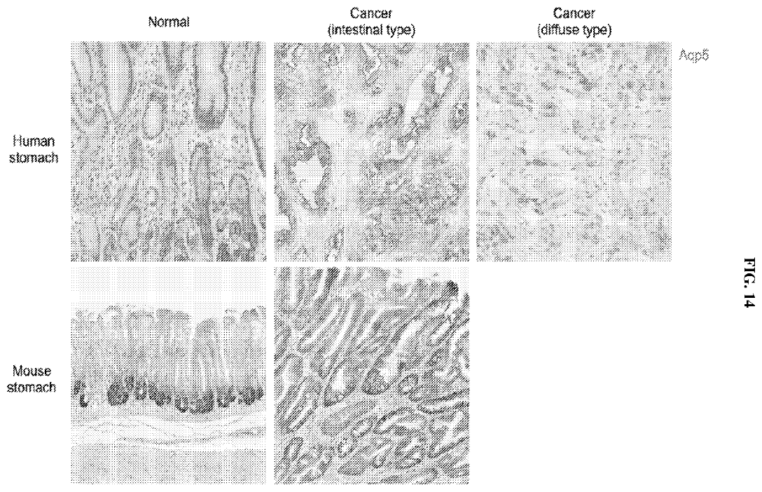

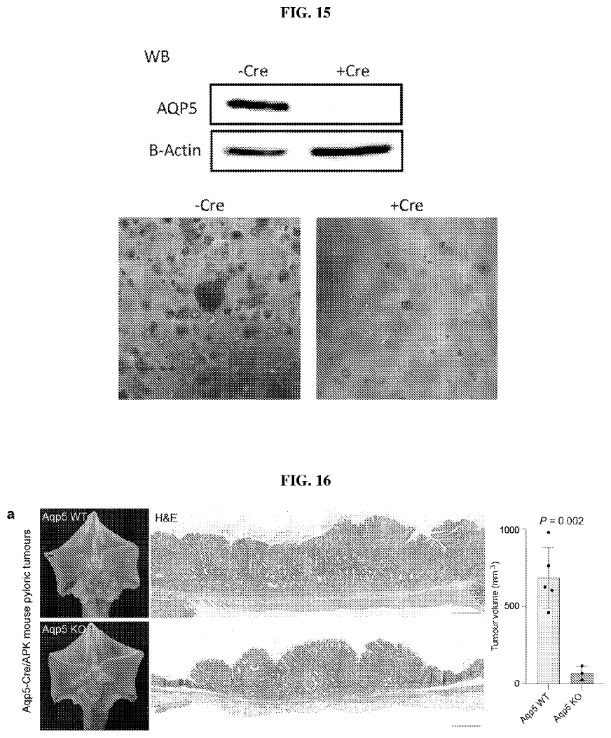

To efficiently ablate Aqp5+ tumour cells in mouse pyloric tumours, the Aqp5-2A-DTR allele was incorporated into the Aqp5-Cre/APK pyloric cancer mouse model (FIG. 10A). The Aqp5-2A-DTR cassette allowed for endogenous expression of Aqp5 together with the diphtheria toxin receptor (DTR) in these Aqp5-expressing cells. Aqp5-Cre/APK/DTR tumours were indistinguishable from Aqp5-Cre/APK tumours in terms of tumour morphology, tumour load, and proportion of tumour-resident Aqp5+ cells (FIG. 10B). To selectively target Aqp5+ pyloric tumour cells, intraperitoneal administration of diphtheria toxin (DT) to Aqp5-Cre/APK/DTR mice harbouring pyloric tumours was performed. Twelve (12) hours after the last DT dose, DT-treated tumours were seen to be significantly reduced in size (FIG. 10C). The loss of Aqp5 expression in DT-treated tumours and the concurrent emergence of Caspase3+ apoptotic cells and Ki67+ proliferative cells in the surviving tumour load (FIG. 10D) was confirmed.

Therefore, in one example, the method disclosed herein further comprises eliminating or ablating the gastric cancer stem cell. In one example, the elimination or ablation is performed using, but not limited to, a compound selected from the group consisting of diphtheria toxin, an inducer of iCasp9 (for example, an agent that dimerizes iCasp9), and an agent that selectively binds to AQP5.

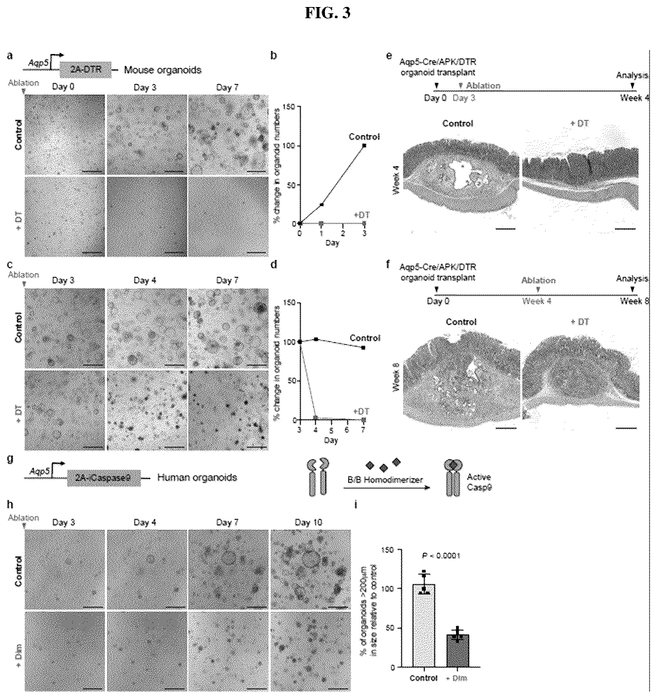

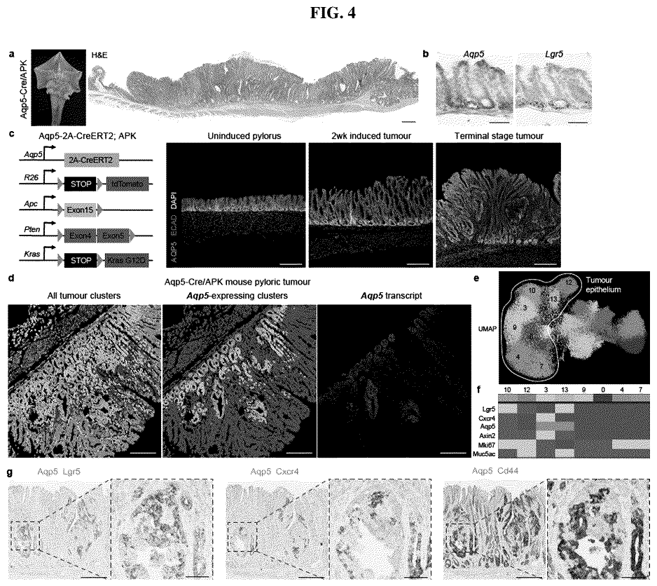

Diphtheria toxin (DT) was not shown to have an effect on pyloric tumours in Aqp5-Cre/APK mice lacking the Aqp5-2A-DTR allele (FIG. 10E), or in Aqp5-Cre/APK and Aqp5-Cre/APK/DTR mice receiving PBS injections without DT (FIG. 10D, FIG. 10E). While administration of DT to Aqp5-Cre/APK/DTR mice effectively ablated Aqp5+ pyloric tumour cells, Aqp5+ cells in other healthy tissues were also affected, leading to high mortality in these mice that precluded the analysis of late tumour timepoints. To circumvent this issue, tumour organoids from Aqp5-Cre/APK/DTR pyloric tumours that maintained a small (9.2%) pool of cells expressing high levels of Aqp5 in culture were generated (FIG. 10F). Addition of DT to Aqp5-Cre/APK/DTR organoid cultures during the initial stages of organoid outgrowth was shown to ablate the majority of Aqp5+ cells, with an 80-90% reduction in Aqp5 levels 1 day after treatment (FIG. 10G) and was also shown to completely abolish organoid outgrowth across four (4) independent lines examined (FIG. 3A, FIG. 3B). The effect of DT administration was also tested on established Aqp5-Cre/APK/DTR organoid cultures, which resulted in a >90% reduction in Aqp5 levels (FIG. 10G) and rapid degeneration of organoids within 1 day of treatment, with no viable organoids remaining after 4 days (FIG. 3C, FIG. 3D). In contrast, the same DT dosage given to Aqp5-Cre/APK organoids lacking the Aqp5-2A-DTR allele had no effect on Aqp5 expression or on organoid outgrowth and subsequent organoid maintenance (FIG. 10H to FIG. 10J). This highlighted a specific effect of Aqp5+ cell ablation on the observed organoid phenotypes.

Thus, in one example, the elimination or ablation is performed using diphtheria toxin.

In yet another example, there is described a method of ablating or eliminating an AQP5+ gastric cancer stem cell that has been modified to express the DTR gene or an inducible Caspase9 (iCasp9) gene, comprising contacting said cell with a DT or an inducer of iCasp9. A non-exhaustive example of such an inducer of iCasp9 is an agent that dimerizes iCasp9.

Also described herein is a method of ablating or eliminating an AQP5+ gastric cancer stem cell comprising contacting the cell with an agent that binds to AQP5, wherein binding of the AQP5+ cell with the agent ablates or eliminates the cell.

To corroborate these findings within a more physiological system, methods to orthotopically transplant Aqp5-Cre/APK/DTR tumour organoids into the pylorus of immune deficient mice, to facilitate the selective ablation of Aqp5+ cells in pyloric tumours developed from these organoids, were established. As the Aqp5-2A-DTR allele is not expressed in other tissues within these mice, it was possible to monitor tumour progression to more advanced stages following DT administration, without complications arising from systemically ablating Aqp5+ cells. Orthotopic transplantation of Aqp5-Cre/APK/DTR pyloric organoids was shown to result in pyloric tumour initiation within 4 weeks, with signs of invasion into the epithelium developing by 8 weeks (FIG. 3E, FIG. 3F). Intraperitoneal injection of DT to mice immediately following orthotopic transplantation of Aqp5-Cre/APK/DTR tumour organoids resulted in the loss of Aqp5-expressing cells (FIG. 10K) and inhibited tumour outgrowth even after 4 weeks (FIG. 3E), across all five (5) independent experiments performed. DT was also administered to established tumours in mice 4 weeks after orthotopic transplantation of Aqp5-Cre/APK/DTR organoids, thereby validating the ablation efficiency in these tumours (FIG. 10L). In contrast to untreated tumours, DT-treated tumours displayed a reduced tumour load with no signs of invasion into the surrounding tissue layers (FIG. 3F). Moreover, it was confirmed that orthotopic tumours derived from Aqp5-Cre/APK tumour organoids without the Aqp5-2A-DTR allele were unaffected by DT administration at both early and late stages of tumour growth (FIG. 10M, FIG. 10N). Taken together, these results indicated a dependence on mouse Aqp5+ tumour cells for maintaining tumour establishment and progression in both organoid and mouse models of pyloric cancer.

The effect of ablating human Aqp5+ cells was tested in a near-physiological human gastric cancer organoid system. To this end, human organoids expressing Aqp5-2AiCaspase, which harboured Aqp5+ cells co-expressing an inducible Caspase9 (iCaspase9) that facilitates the selective induction of caspase-mediated cell death within Aqp5+ cells upon administration of a dimerizing agent (FIG. 3H, FIG. 3I and FIG. 11C), were generated. The resulting Aqp5-2A-iCaspase organoids presented comparable organoid morphology, Aqp5 expression, and cell lineage marker expression compared to non-edited tumour organoids (FIG. 11B).

Thus, in one example, the cell or cell population disclosed herein is modified to express AQP5 in conjunction with a diphtheria toxin receptor (DTR) gene or an inducible Caspase9 (iCasp9) gene.

As described herein, Aqp5-2A-iCaspase9 constructs were generated for expression in cells. This means that cells that express Aqp5 will simultaneously produce iCaspase9. Upon addition of an inducer such as, for example, AP20187 (dimerizer), iCaspase9 undergoes homodimerization to generate the functional protein, resulting in apoptosis specifically in Aqp5-expressing cells, while leaving non-Aqp5-expressing cells intact.

To ablate human Aqp5+ cells, a B/B homodimerizer agent was administered to Aqp5-2AiCaspase human organoid cultures. This resulted in a loss of 60% in Aqp5 expression in treated organoids (FIG. 11C). These treated Aqp5-2A-iCaspase tumour organoids also displayed diminished growth rates in culture (FIG. 3H, FIG. 3I), although they continued to be viable. Without being bound by theory, this was thought to be due to the remaining unablated Aqp5+ cells in these cultures. Non-edited tumour organoids were unaffected by the treatment with the B/B homodimerizer, with no significant differences in both Aqp5 levels and organoid sizes observed (FIG. 11D). Ablation experiments were performed in an independent human gastric cancer organoid line, GC10, thus confirming the specific effect of ablating Aqp5+ cells on organoid growth (FIG. 11E to FIG. 11H). Like their mouse counterparts, human Aqp5+ cancer stem cells in tumour organoids were shown to also be a key driver of organoid growth, thereby underlining their effectiveness as a therapeutic target to drive cancer regression.

The cancer stem cell model links clinical observations of chemoresistance and tumour relapse to the activity of a dedicated pool of stem cells in fueling continued tumour growth, which can then be harnessed for therapeutic use. While the cancer stem cell theory has been substantiated in a number of blood, brain, and colon cancers, the presence of such a stem cell pool within gastric tumours had yet to be robustly proven through multiple assays of stem cell activity using physiologically relevant mouse and human gastric cancer models. It is shown here that both mouse and human pyloric tumours harbour a distinct Aqp5+ cancer stem cell population capable of initiating long-term organoid cultures and re-establishing invasive pyloric tumours when transplanted into the mouse stomach. Targeted ablation of Aqp5+ cells was also shown to be sufficient to block pyloric tumour initiation and growth, establishing the central role played by these cells in disease progression. These findings showed that gastric tumours can be driven by an Aqp5+ stem population, thereby highlight capability of using Aqp5+ cancer stem cells as a target in the treatment of gastric cancer.

Also contemplated in the scope of the present disclosure is use of Aqp5 as a gastric cancer stem cell marker that is membrane-bound and amenable to FACS sorting. This application facilitates the specific isolation of these gastric cancer stem cells directly from native tumours, thereby enabling an in-depth study of cancer stem cell biology and mechanisms driving their tumourigenic behaviours. This includes the identification of pathways promoting cancer stem cell functions in these Aqp5-expressing cells as well as other cancer stem cell-specific genes that represent targets that can be used to eliminate or perturb the functions of Aqp5-expressing cell populations.

Thus, in one example, the method of detecting, isolating, or identifying a gastric cancer stem cell as disclosed herein comprises detecting, isolating, or identifying membrane bound AQP5. In other words, in another example, the AQP5 disclosed herein is membrane-bound.

Thus, the present disclosure also contemplates a method of treating gastric cancer in a subject in need thereof. In one example, the method of treating gastric cancer in a subject identified to have gastric cancer stem cells or gastric cancer can comprise a step of administering a therapy or compound, that is in line with the standard of care treatment for gastric cancer, to a subject. Examples of standard of care can be, but is not limited to, capecitabine, cyramza (ramucirumab), docetaxel, doxorubicin hydrochloride, enhertu (Fam-trastuzumab deruxtecan-nxki), 5-FU (fluorouracil injection), fam-trastuzumab deruxtecan-nxki, fluorouracil, herceptin (trastuzumab), keytruda (pembrolizumab), lonsurf (trifluridine and tipiracil hydrochloride), mitomycin, nivolumab, opdivo (nivolumab), pembrolizumab, ramucirumab, taxotere (docetaxel), trastuzumab, trifluridine and tipiracil hydrochloride, xeloda (capecitabine), resection/surgery/surgical resection, endoscopic mucosal resection, perioperative chemotherapy without or without radiation therapy, radiation therapy, postoperative (adjuvant) chemoradiation therapy, postoperative (adjuvant) chemotherapy, and combinations thereof. As can be appreciated by a person skilled in the art, treatments may vary depending on the stage or progression of the disease, once identified.

In one example, the method comprises administering a therapeutically effective amount of one or more agents that eliminates or ablates AQP5-expressing cells to the subject. In another example, the treating includes curing the gastric cancer, reducing growth of the gastric cancer, slowing the progression of the gastric cancer, improving prognosis of the subject or combinations thereof. In another example, there is disclosed the use of one or more agents that eliminates or ablates AQP5-expressing cells in the manufacture of a medicament for treating gastric cancer. In another example, one or more agents that eliminates or ablates AQP5-expressing cells for use in therapy are disclosed. In a further example, one or more agents that eliminates or ablates AQP5-expressing cells for use in treating gastric cancer are disclosed.

In one example, the subject is a human or mouse. In another example, the subject is human.

Also disclosed herein are one or more agents that eliminates or ablates AQP5-expressing cells for use in therapy. In another example, one or more agents that eliminates or ablates AQP5-expressing cells are disclosed for use in treating gastric cancer.

The methods disclosed herein can also be used to develop approaches to recognise, target, and eliminate Aqp5-expressing gastric cancer stem cells within the tumour load to curb or halt cancer progression. The detection of Aqp5-expressing gastric cancer cells within tumours can also aid in cancer diagnostics and serve as an indicator of disease stage/severity.

Also contemplated herein is a kit for identifying, isolating, eliminating or ablating a gastric cancer stem cell. In one example, the kit described herein comprises an agent that binds to AQP5, and instructions for use. In another example, the kit further comprises an antibody, such as a detection antibody. In another example, the antibody is conjugated to a compound such as a detectable label, a drug, or a small molecule. In another example, the antibody binds to the intracellular domain or extracellular domain of the AQP5 protein.

AQP5 has been identified as a major driver of gastric cancer progression across multiple near-physiological mouse and human models of intestinal and diffuse-type gastric cancer. In gastric cancer patients, AQP5 is broadly overexpressed across multiple subtypes/stages of gastric tumours and their associated metastases relative to healthy gastric tissues, highlighting a potential cancer-specific role of Aqp5 in driving the tumourigenic state. Indeed, knocking out Aqp5 in our mouse and human gastric cancer models resulted in significant reductions in tumour loads and, in some cases, completely eliminated cancer initiation. Conversely, AQP5 overexpression accelerates the development of tumour features. Targeting Aqp5 expression and/or its downstream functions represents a promising new direction in the treatment of gastric cancer.

Gastric cancer is one of the leading causes of cancer-related deaths worldwide and in Singapore, with a five-year survival rate of less than 30%. Treatment of gastric cancer is currently limited to traditional methods of chemotherapy, radiation therapy, and surgical resection, but these approaches remain ineffective in ameliorating cancer relapse in many gastric cancer patients. There is thus a need to evaluate new treatment modalities, such as by targeting novel functional regulators of cancer initiation, progression and metastasis.

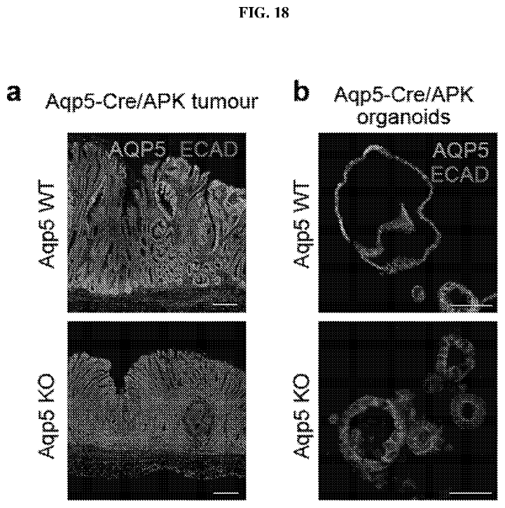

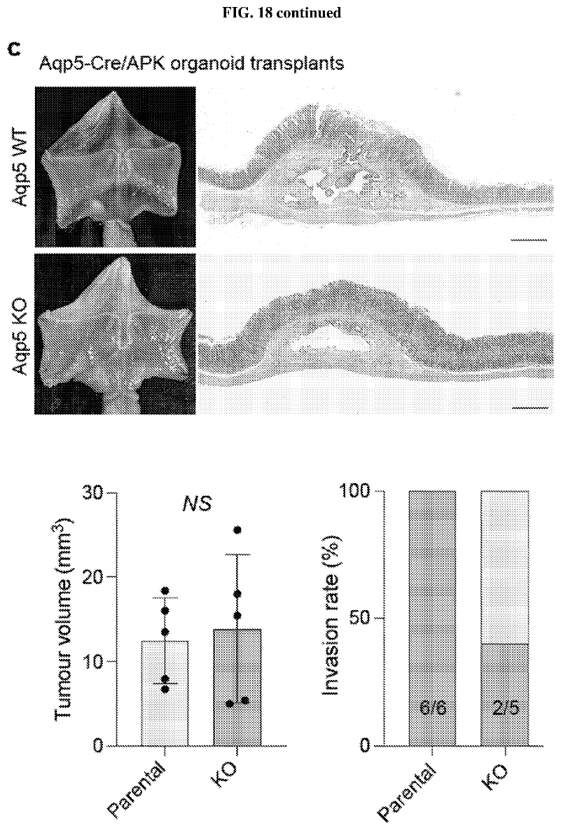

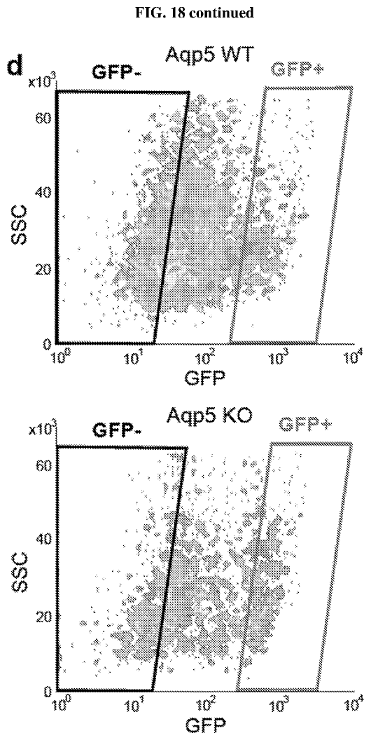

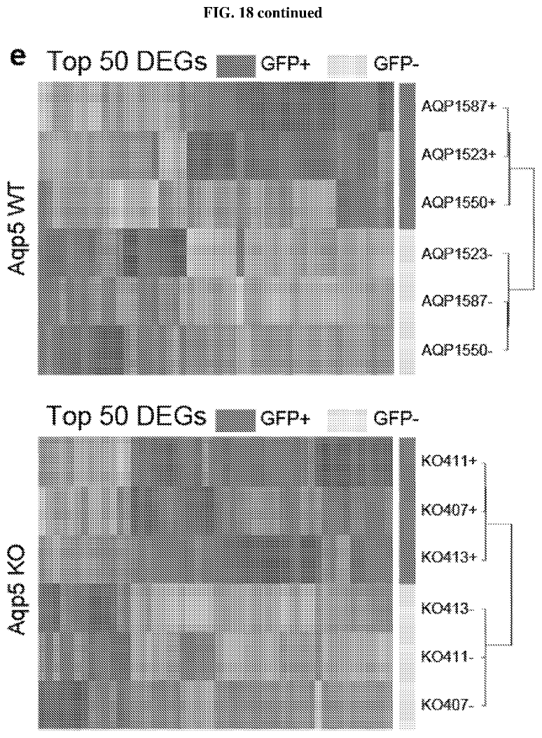

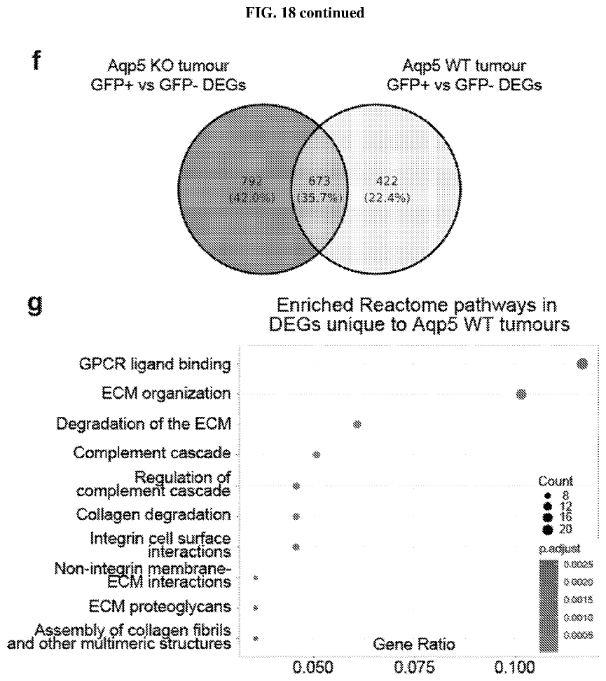

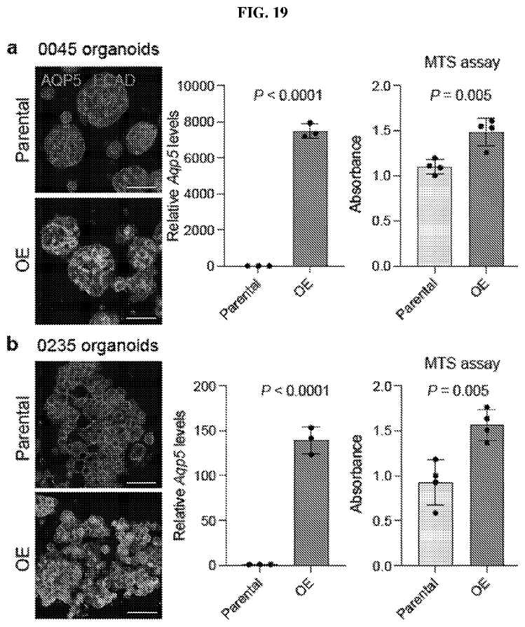

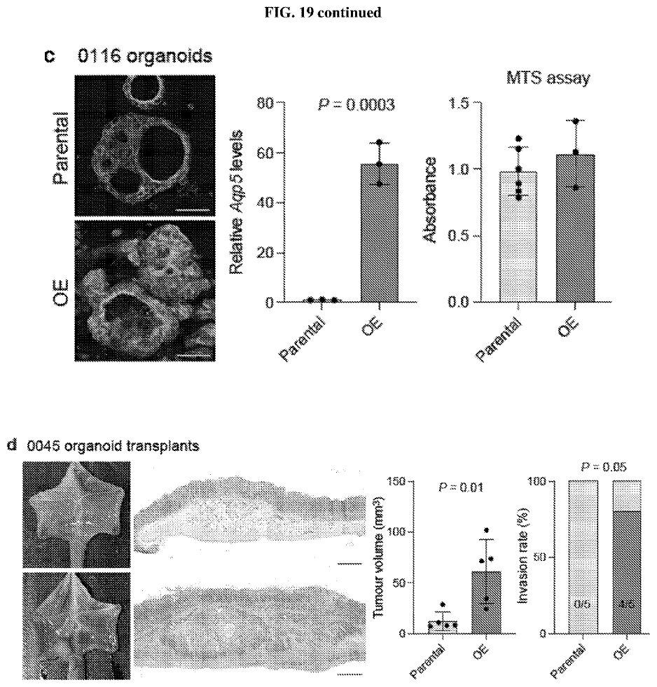

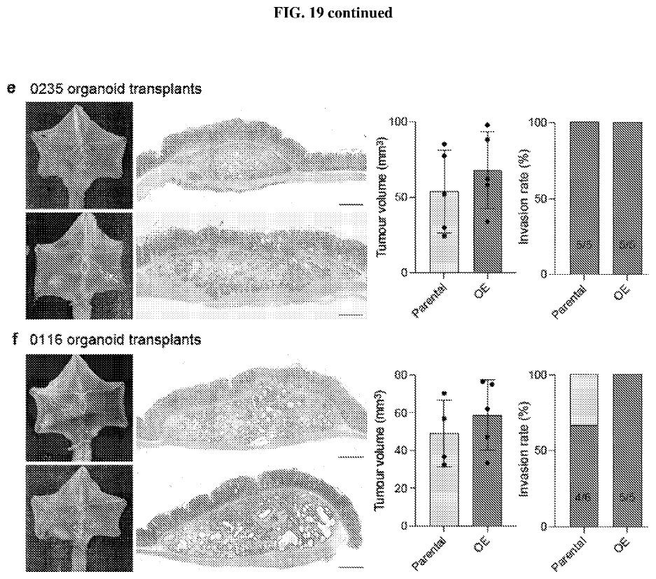

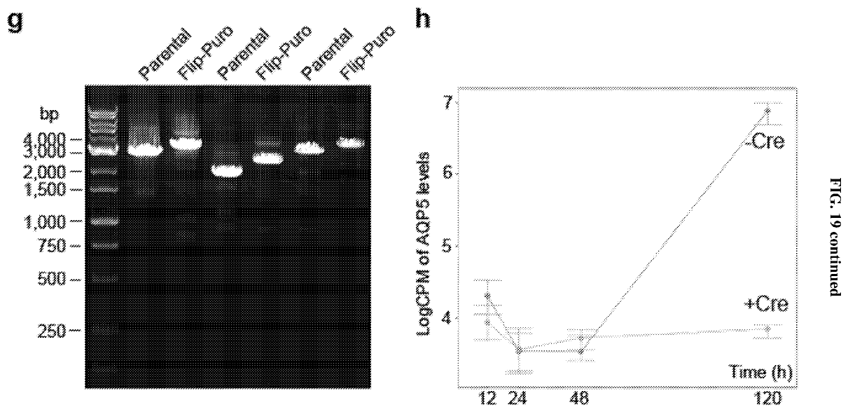

Aqp5 has been identified herein as a major driver of gastric cancer progression across multiple near-physiological mouse and human gastric cancer model systems. In gastric cancer patients, Aqp5 is frequently overexpressed in both intestinal- and diffuse-type tumours relative to healthy gastric tissue, a phenotype also mirrored in healthy and cancerous murine gastric tissues (FIG. 17 and FIG. 18). Using a panel of human gastric cancer cell lines, it is shown that both intestinal-type and diffuse-type cancer cell lines present elevated levels of cell proliferation and migration in vitro when Aqp5 is overexpressed. Moreover, following orthotopic transplantation of these human gastric cancer cell lines into the pyloric submucosa of immune-deficient mice, Aqp5-overexpressing lines seed larger tumours that display more aggressive histological features, including high levels of cell proliferation and invasion into adjacent tissues (FIG. 19). It was further shown that these tumour phenotypes can be replicated across independent human gastric cancer organoid lines. These three-dimensional organoid models are grown within an extracellular matrix-like environment that preserves morphological and molecular features resembling the native epithelium, in contrast to two-dimensional cell line monolayer cultures. Human gastric cancer organoids xenografted into the pylorus of immune-deficient mice were able to seed pyloric tumours, and Aqp5-overexpressing organoids established larger, more aggressive tumours that frequently invaded through the pyloric epithelium and external muscle layers. This work thus establishes Aqp5 as a pervasive driver of gastric cancer tumourigenesis across multiple gastric cancer types and models.

To further evaluate Aqp5 functions and the potential of a therapeutic strategy targeting Aqp5 expression, Aqp5 knockout gastric cancer models were established and assessed the effect of Aqp5 loss on gastric tumourigenesis (FIG. 20).

In human gastric cancer cell lines lacking Aqp5, cells proliferated more slowly and had severely impaired abilities in seeding tumours when orthotopically transplanted into immune-deficient mice. Similarly, human gastric cancer organoids with CRISPR/Cas9-mediated knockout of Aqp5 initiated tumours in mice that were significantly smaller and failed to invade into surrounding tissue layers. These findings confirm the role of Aqp5 in driving tumourigenesis and highlight the potential of eliminating Aqp5 as a way to block tumour progression and possibly promote its regression.

Finally, a Aqp5 knockout mouse gastric cancer model was established in which tumours can be selectively generated within the pylorus of the stomach upon the targeted recombination of cancer-inducing alleles. Aqp5 knockout mice are phenotypically normal and do not display altered gastric functions, but upon induction of tumour formation, these mice develop smaller pyloric tumours. Moreover, it was shown that isolated gastric cancer stem cells from Aqp5 knockout tumours had reduced stem potential, generating fewer organoids in culture compared to their Aqp5 wildtype counterparts. In all, it is shown that Aqp5 plays a specific role in the gastric cancer context to drive tumourigenic properties that promote the onset and progression of the disease. Targeting Aqp5 expression and/or regulators of its expression and functions is therefore a viable therapeutic approach for gastric cancer patients.

Thus, in one example, there is disclosed a method of inhibiting gastric cancer tumorigenesis or gastric cancer progression, the method comprising administering an AQP5 inhibitor to a subject. In another example, there is disclosed a method of promoting gastric cancer regression, the method comprising administering an AQP5 inhibitor to a subject.

In one example, the AQP5 inhibitor is a compound that inhibits or blocks AQP5 expression and/or function. Examples of AQP5 inhibitors include, but are not limited to an siRNA, an RNAi, a chimeric antigen receptor (CAR), and a drug.

In one example, the AQP5 inhibitor is an inhibitory RNA (RNAi). Examples of RNAi include, but are not limited to, siRNA, shRNA, and miRNA. In another example, the AQP5 inhibitor is an siRNA. In one example, the siRNA can be, but is not limited to, a sense and antisense primer pair. In another example, the primer is, but is not limited to 5′-AAAACTCTGCGAACACGGCCCCTGTCTC-3′ (SEQ ID NO: 49) and 5′-AAGGCCGTGTTCGCAGAGTTCCTGTCTC-3′ (SEQ ID NO: 50); 5′-CGGUGGUCAUGAAUCGGUUTT-3′ (SEQ ID NO: 51) and 5′-AACCGAUUCAUGACCACCGCA-3′ (SEQ ID NO: 52); and 5′-GCGUGUGGCCAUCAUCAAATT-3′ (SEQ ID NO: 53) and 5′-UUUGAUGAUGGCCACACGCTT-3′ (SEQ ID NO: 54); and combinations thereof.

In one example, the drug is, but not limited to, enzymatic inhibitors, receptor antagonists, and channel blockers.

A functional driver of gastric cancer progression was found to accelerate development of tumourigenic features when overexpressed. In contrast, knocking out this driver impairs gastric cancer progression and is sufficient to block cancer initiation in some contexts. Thus, it is shown that targeting the expression of AQP5 and/or its functional regulators can serve as a therapeutic approach for the treatment of gastric cancer.

As shown herein, multiple Aqp5 knockout and Aqp5 overexpressing mouse and human gastric cancer cell line, organoid, and xenograft mouse models have been generated. Through these models, Aqp5 functions across independent systems and multiple gastric cancer subtypes have been validated, highlighting the widespread relevance of our findings to the majority of gastric cancer patients. Moreover, without being bound by theory, the Aqp5 knockout gastric cancer genetic mouse model disclosed herein allows for targeted induction of pyloric tumour formation within the mouse. This provides insights into Aqp5 functions across the entire temporal spectrum of the disease from onset to tumour spread within a native tissue context. The technology disclosed herein therefore robustly proves Aqp5 functions in gastric tumourigenesis across independent, near-physiological models that capture the diversity of human gastric tumour types, and highlights the utility of targeting Aqp5 and/or regulators of Aqp5 expression and functions as a therapeutic approach for the treatment of gastric cancer.

In one example, there is described a method of monitoring progression of a gastric cancer in a subject, the method comprising a) measuring the expression level of AQP5 in a sample obtained from the subject after having undergone treatment for gastric cancer; and b) measuring the expression level of AQP5 in a control sample obtained from the subject prior to treatment for gastric cancer; wherein an increase in the expression level of AQP5 in the sample of step a) compared to the control sample indicates that gastric cancer tumorigenesis has taken place or that the gastric cancer has progressed.

In another example, there is described a method of monitoring gastric cancer tumorigenesis in a subject, the method comprising c) measuring the expression level of AQP5 in a sample obtained from the subject; and d) measuring the expression level of AQP5 in a reference sample obtained from the subject at a timepoint earlier than the sample of step c; wherein an increase in the expression level of AQP5 in the sample of step c compared to the reference sample of step d indicates that gastric cancer tumorigenesis has taken place.

In one example, the expression level of AQP5 is gene expression level, protein expression level or a combination thereof. In another example, the method described herein further comprises measuring the expression levels of one or more markers of gastric cancer progression. In another example, the expression levels of one or more markers of gastric cancer progression are measured in addition to the other expression levels disclosed herein. Examples of markers of gastric cancer progression can be, but are not limited to, Pthlh, Hey1, Rgs5, and combinations thereof.

In one example, if gastric cancer tumorigenesis has taken place or that the gastric cancer has progressed, the subject is to be treated with an anti-gastric cancer compound or a standard of care treatment for gastric cancer.

A person skilled in the art would be able to discern which methods can be used to measure the levels disclosed herein. In one example, the step of measuring the expression level of AQP5 is by immunohistochemistry, flow cytometry, Western blot, or combinations thereof. In another example, the step of measuring the expression level of AQP5 is by polymerase chain reaction.

In one example, the expression levels are measured in a sample. Examples of such samples are, but are not limited to, blood, blood plasma, biopsy sample, tissue sample, primary cell culture sample, and primary organoid lines.

The methods disclosed herein relate to gastric cancer. Gastric cancer, also referred to as stomach cancer, is a cancer which develops in the gastric mucosa. Gastric cancers can include, but are not limited to, adenocarcinomas, lymphomas and mesenchymal tumours. In one example, the gastric cancer is, but is not limited to, intestinal-type gastric cancer or diffuse-type gastric cancer.

It is challenging to identify specific genes solely expressed in tumours but completely absent from normal tissues. Indeed, AQP5 is not only expressed in gastric tumours but also present in a small proportion of healthy tissues, including lungs, salivary glands and testes. Levels of AQP5 are minimal/low and primarily cytoplasmic in many of these healthy tissues, including the lungs. Moreover, mice lacking AQP5 (for example, full knockout mice) are viable and healthy with only a minor defect in saliva secretion documented in these animals, suggesting that Aqp5 may not play a critical role outside the cancer state. Life-threatening consequences from a targeted approach to eliminate AQP5 or its regulators are not expected, therefore, an appropriate therapeutic window can be calibrated to ensure effective targeting of the AQP5-expressing gastric tumour cells, while posing minimal damage to other AQP5-expressing healthy tissues.

This technology facilitates the development of approaches to recognize, target, and eliminate AQP5 and/or regulators of AQP5 expression and functions within gastric tumours as a means to curb cancer progression. Moreover, the detection of high AQP5 levels within gastric tumours serves as an indicator of disease stage/severity and aid in cancer diagnostics. The suite of in vitro and in vivo gastric cancer models and the AQP5 knockout gastric cancer genetic mouse model generated herein can also be used in drug testing efforts to evaluate novel therapeutics aimed at ameliorating disease progression. Finally, this technology can enable identifying other protein partners of AQP5 and the immediate downstream pathways regulated by AQP5 that drive tumour progression, expanding the list of targetable components and scope of therapeutic approaches available for gastric cancer patients.

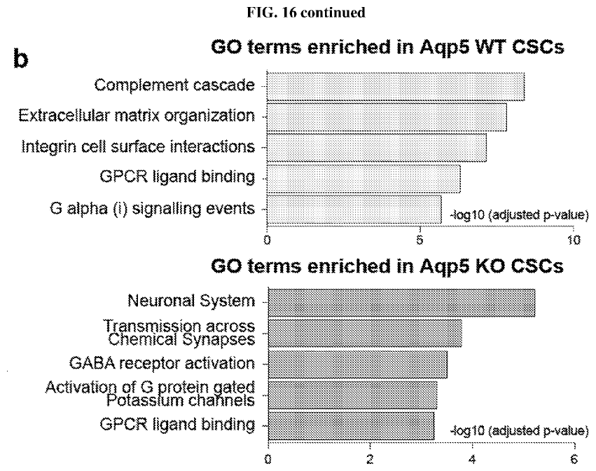

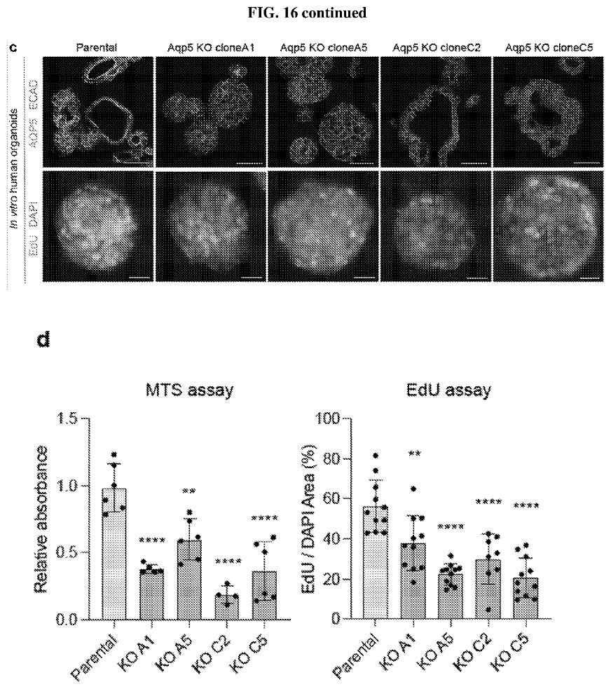

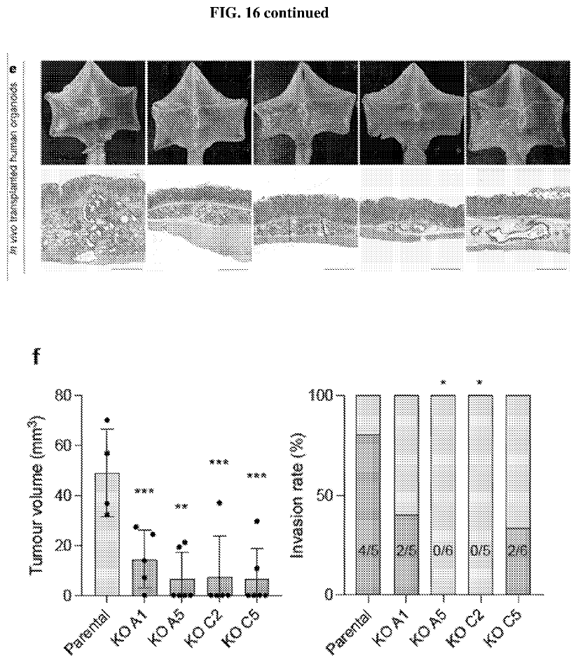

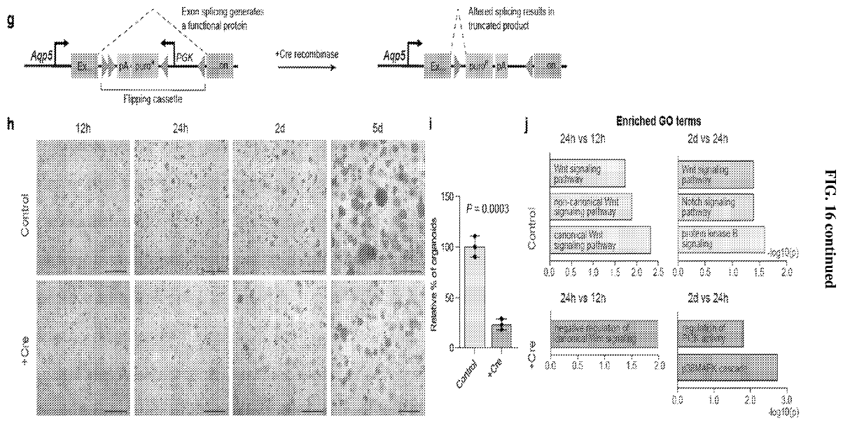

The experimental data as shown in FIGS. 16 to 20, using multiple near-physiological gastric cancer models, underline the central role played by AQP5 in driving gastric cancer. AQP5 knockout gastric cancer cells are shown to be less proliferative and form smaller, less aggressive tumours in vivo. Conversely, overexpression of AQP5 in gastric cancer cells increases cell proliferation and migration in vitro and accelerates tumour progression in vivo. Finally, using the AQP5 knockout gastric cancer mouse model disclosed herein, it is shown that in the complete absence of AQP5, mice developed smaller precancerous neoplastic lesions instead of the fully-grown gastric adenocarcinomas typically observed in such cancer mouse models.

A further, previously unknown human gastric cancer organoid line integrated with a Flip-Puro system enabling conditional knockout of AQP5 upon administration of Cre recombinase gesicles has been generated. Loss of AQP5 in these organoids was confirmed by western blotting. Reduced cell viability of organoids following AQP5 knockout was found to be present, as shown in FIG. 21.

### Aqp5 Drivers Tumour Progression in Mouse and Human Models of Gastric Cancer

An Aqp5 knockout pyloric tumour mouse model (Aqp5KO-APK) by incorporating an Aqp5 null allele alongside the Aqp5-eGFP-IRES-creERT2 cassette (which resulted in inactivation of both copies of Aqp5). Prior to cancer induction, Aqp5 null mice are healthy and indistinguishable from their Aqp5 wildtype counterparts. Following tamoxifen administration, pyloric tumours formed in these Aqp5KO-APK mice with complete absence of Aqp5 expression (FIG. 16A, FIG. 18A). Crucially, these Aqp5-null tumours were also significantly smaller in volume and presented pre-cancerous features of intraepithelial neoplasia, whereas wildtype tumours were classified as adenocarcinomas by the same timepoint (FIG. 16A), indicating that Aqp5 could play a role in driving tumour progression in an in vivo context. To replicate these findings in vitro, ted Aqp5-null mouse pyloric tumour organoids from Aqp5KO-APK tumours and confirmed that they also fail to express Aqp5 (FIG. 18B). In agreement with Aqp5 knockout in native tumours, Aqp5KO tumour organoids formed tumours following orthotopic transplantation that presented less invasive phenotypes (FIG. 18C), highlighting the role of Aqp5 in driving tumour progression in this context.

To further dissect the functions of Aqp5 in the stem cell compartment of Aqp5KO-APK pyloric tumours, the GFP marker, whose expression is driven by the Aqp5 promoter, was used to isolate the putative cancer stem cells (GFP+) and the remaining tumour epithelial bulk (GFP−) from both Aqp5 knockout and Aqp5 wildtype mouse pyloric tumours for bulk RNA sequencing (FIG. 18D, FIG. 18E). This yielded a cancer stem cell signature including genes common to both Aqp5 wildtype and knockout tumours (presumably genes unaffected by Aqp5 levels) as well as differentially expressed genes unique to Aqp5 wildtype and knockout tumours (FIG. 18F). Pathway analyses indicated that the genes unique to Aqp5 wildtype cancer stem cells were enriched in ECM organization and G protein signalling pathways (FIG. 16B, FIG. 18G). These profiling studies identified pathways that could function downstream of Aqp5 within the cancer stem cell compartment in driving tumour progression. Aqp5 functions were then probed in human gastric cancer organoids, which offer greater cellular complexity and resemblance to primary tumours than cancer cell lines. Four independent Aqp5 knockout organoid clones were identified and verified the loss of Aqp5 expression in these lines (FIG. 16C). All four Aqp5-null organoid lines reproducibly showed lower rates of proliferation in vitro relative to the Aqp5 wildtype parental organoid line (FIG. 16D) and established smaller tumours following orthotopic transplantation into mice that were frequently less invasive (FIG. 16E, FIG. 16F). Conversely, overexpression of Aqp5 was sufficient to increase cell proliferation in vitro in two additional human gastric cancer organoid lines (FIG. 19A, FIG. 19B), although there was no significant increase when Aqp5 overexpression was performed in an organoid line already expressing high levels of Aqp5 (FIG. 19C), suggesting that Aqp5-induced phenotypes could be dose-dependent up to a fixed threshold. Following orthotopic transplantation, Aqp5-overexpressing human organoids established larger tumours that were also more invasive in vivo (FIG. 19D, FIG. 19E), but these tumour differences were again negligible in a line already expressing high Aqp5 levels (FIG. 19F).

Finally, to dissect the mechanisms driving Aqp5 functions in human gastric cancer, human organoids amenable to conditional knockout of Aqp5 based on the FLIP-Puro system (FIG. 19G) were generated. Administration of Cre recombinase gesicles resulted in altered exon splicing that generates a truncated, non-functional Aqp5 product (FIG. 1G). It was confirmed that Cre gesicle-treated human organoids no longer expressed Aqp5 and grew more slowly in culture (FIG. 16H, FIG. 16I, FIG. 19H), concurring with the phenotypes observed from the constitutive Aqp5 KO human organoid lines (FIG. 16C, FIG. 16D). Bulk RNAseq on was then performed on Aqp5 FLIP-Puro organoids at several timepoints immediately following Cre recombination and performed pathway analyses on the generated transcriptomic datasets. This revealed a multitude of pathways activated at early stages of organoid growth related to Wnt signaling, which were abolished immediately following Aqp5 knockout (FIG. 22J), highlighting a potential mechanism of action for Aqp5 functions in gastric cancer.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Mouse Work

Aqp5-eGFP-ires-creERT2, Aqp5-2A-creERT2, and Aqp5-2A-DTR mice had been previously generated. Mice containing Rosa26-tdTomato^{LSL} (Ai14) (JAX 007914), Apc^{fl/fl} (MGI 1857966), Pten^{fl/fl} (MGI 2182005), Kras^{LSL-G12D} (JAX 019104), and p53^{R172H} have also been previously described. AQP5 knockout (KO) mice were obtained from Cyagen (KOCMP-11830-Aqp5-B6N-VA). For induction of Cre recombination, 8-week-old mice were injected intraperitoneally with 4 mg Tamoxifen (Merck T5648) dissolved in sunflower oil per 30 g of mouse weight. Diphtheria toxin (DT; Sigma D0564) administration was performed intraperitoneally, at a dose of 0.5 μg DT dissolved in PBS per 30 g of mouse weight.

Orthotopic transplants were performed on NOD.Cg-Prkdc^{scid} II2rg^{tm1Wjl}/SzJ (NSG) mice (JAX 005557). Briefly, mice were anesthetised, and their stomach exposed under sterile surgical conditions. Injection was performed using an insulin syringe containing the transplantation material (cells or organoids) inserted in the pylorus region immediately beneath the outer muscle layer. Following the procedure, the wounds were sutured, and the mice administered the reversal drug Atipamezole, along with Buprenorphine over several days after surgery.

All mouse experiments were performed in accordance with ethical and safety regulations covered by the Institutional Animal Care and Use Committee (IACUC) and A*STAR. Mice were immediately sacrificed when tumours reached the maximum allowable size of 20 mm.

### Human Samples

Patient pyloric tumour biopsies were provided by J. So, Department of Gastroenterology, National University Hospital Singapore (IRB protocol 2020-038). Informed consent was obtained from all patients. Tumour samples were collected in an Advanced DMEM/F-12 media (Invitrogen 12634-028) with 2 mM Glutamax (Invitrogen 35050-079), 10 mM HEPES (Invitrogen 15630-056), 1 mM N-acetylcysteine (Sigma A9165), and 1× Antibiotic-Antimycotic (Gibco 15240096). Samples were washed vigorously in HBSS prior to proceeding with steps for tissue dissociation. All experiments involving human material were performed in accordance with ethical and safety regulations covered by the A*STAR Institutional Review Board (IRB).

### Tissue Dissociation for FACS

Dissociation of pyloric tumours was performed by incubation of minced tissue in an Advanced DMEM/F-12 media (Invitrogen 12634-028) with 2 mM Glutamax (Invitrogen 35050-079), 10 mM HEPES (Invitrogen 15630-056), 2 mg/ml Bovine Serum Albumin (BSA), and 1 mg/ml Collagenase Type I (Life Technologies 17100017), at 37° C. for 45 minutes. To release the gastric glands, minced tissues were vigorously and repeatedly pipetted using cold Advanced DMEM/F-12 buffer and filtered through a 100 μm filter mesh before being spun down at 2500 rpm at 4° C. for 3 minutes. The resultant pellet was further dissociated into a single cell suspension by incubating with TrypLE (Gibco 12604) and 10 mg/ml DNase I (Sigma D4513) at 37° C. for 10 minutes. The digestion reaction was quenched using 30 ml cold HBSS, and the suspension centrifuged at 2500 rpm at 4° C. for 3 minutes.

For mouse pyloric tumours with AQP5 tagged with eGFP, the cell pellet was directly resuspended in 2% fetal bovine serum (FBS) in HBSS and passed through a 40 μm filter for cell sorting on the BD Influx Cell Sorter (BD Biosciences). For human pyloric tumours, single cell suspensions following TrypLE digestion were incubated with AQP5-AF647 (1:500, Abcam ab215225), CD11b-FITC (1:200, Biolegend 101205), CD31-FITC (1:200, Biolegend 303103), and CD45-FITC (1:200, Biolegend 304054) in 2% FBS in HBSS at 4° C. for 45 minutes.

Cells were washed twice with 2% FBS in HBSS prior to filtering through the 40 μm filter for FACS. Prior to FACS, cells were resuspended with DAPI for live/dead cell gating. Sorted cells were collected directly in RLT Plus Buffer (Qiagen) with b-mercaptoethanol for RNA isolation or Advanced DMEM/F-12 with 0.5% Matrigel (Corning 354253) for organoid culture.

### Organoid Culture

Pyloric tumour organoids were grown in a basal media comprised Advanced DMEM/F-12 (Invitrogen 12634-028) with 2 mM Glutamax (Invitrogen 35050-079), 10 mM HEPES (Invitrogen 15630-056), 1× N2 (Invitrogen 17502-048), 1× B27 (Invitrogen 17504-044), 1 mM N-acetylcysteine (Sigma A9165), and 200 μg/ml Primocin (InvivoGen Ant-pm-1). For mouse pyloric tumour organoid cultures, organoids in their first passage after plating were supplemented with these recombinant growth factors at the indicated concentrations: 50 ng/ml EGF (Invitrogen PMG8043), 100 ng/ml FGF10 (Peprotech 100-26), 10 nM GAST (Sigma G9145), 100 ng/ml WNT3A (Peprotech 315-20), 1 μg/ml RSPO1 (Peprotech 120-38), 100 ng/ml NOGGIN (Peprotech 250-38). Following establishment, the cultures were switched to basal media for long-term maintenance to select for tumour organoids. Human gastric cancer organoids HCM-BROD-0045-C16, HCM-BROD-0116-C16, and HCM-BROD-0235-C16 were purchased from ATCC. Human organoid cultures were given the abovementioned recombinant growth factors along with 2 μM A-8301 (Tocris 2939) and 10 mM Nicotinamide (Sigma N0636) in a base media composed of 50% WRNF conditioned media. Single cell cultures also received 10UM Y-27632 (Tocris 1254). Organoids were passaged when confluent approximately once a week by dissociating them with TrypLE (Gibco 12604) and continued maintenance in Growth Factor Reduced Matrigel basement membrane matrix (Corning 356231). For experiments involving orthotopic transplantation of organoids into mice, intact organoids were retrieved from Matrigel using Cell Recovery Solution (Corning 354253).

To generate gene-edited human gastric cancer organoids, the following plasmids were electroporated: HR110PA-1 hAQP5-5′arm-P2A-iCas9-IRES-Venus-3′arm, pX330 hAQP5 gRNA-1, and pX330 hAQP5 gRNA-2 to generate Aqp5-2A-iCaspase organoids, and HR110PA-1 hAQP5-5′arm-CreERT2-pA-3′arm, pX330 hAQP5 gRNA-1, and pX330 hAQP5 gRNA-2 followed by AAVS1-2A-Blasticidin-CAG-LSL-tdTomato and pX330 AAVS1 gRNA to generate Aqp5-2A-CreERT2; LSL-tdTomato organoids. The pX330 plasmids were derived from pX330-U6-Chimeric_BB-CBh-hSpCas9 (Addgene plasmid #42230 from Feng Zhang). Electroporation of organoids was performed using the NEPA21 Electroporator (NEPA GENE) following manufacturer's recommendations. Briefly, organoids were supplemented with 5 μM CHIR99021 (Stemgent 04-0004-10) and 10 μM Y-27632 (Tocris 1254) one day prior to electroporation. Organoids were dissociated using TrypLE (Gibco 12604) and washed with Opti-MEM I reduced serum medium (Life Technologies 31985-062). The cell pellet was resuspended with BTXpress (BTX Harvard Apparatus 45-0805) and the respective plasmids at a ratio of 100,000 cells to 15 μg DNA before loading into the electroporation cuvette. Electroporation was performed with a poring pulse of 175 V and pulse length of 5 milliseconds. Successful transfectants were selected by puromycin (InvivoGen Ant-pr-1), with the exception of the AAVS1-2A-Blasticidin-CAG-LSL-tdTomato organoids selected by blasticidin (InvivoGen Ant-bl-1). Following transfection of the Aqp5-2A-iCaspase and Aqp5-2ACreERT2 constructs, organoids were further treated with Cre Recombinase Gesicles (Clontech 631449) to remove the selection cassette. Briefly, organoids were dissociated by TrypLE (Gibco 12604) and washed with 10% FBS in Advanced DMEM/F12 (Invitrogen 12634-028). Cre Recombinase Gesicles (Clontech 631449) and 6 μg/ml polybrene (Sigma H9268) were added and the suspension centrifuged at 600 g at 32° C. for 1 hour. The cells were incubated at 37° C. for 6 hours before transferring into Matrigel. Following Cre Gesicle treatment, cells were grown in organoid media without puromycin for 1 week and cell sorting performed on the BD Influx Cell Sorter (BD Biosciences) to collect RFP negative cells.

To induce Aqp5+ cell ablation, organoids were treated with 500 nM B/B Homodimerizer (Clontech 635059) for Aqp5-2A-iCaspase organoids and 240 ng/ml DT for Aqp5-2A-DTR organoids. To perform lineage tracing of Aqp5+ cells in Aqp5-2A-CreERT2; LSL-tdTomato organoids, 1 μM 4-OHT was administered to organoids for 16 hours and removed thereafter. For conditional Aqp5 KO in Aqp5-FLIP-Puro organoids, organoids were dissociated by TrypLE (Gibco 12604) and washed with 10% FBS in Advanced DMEM/F12 (Invitrogen 12634-028). Cre Recombinase Gesicles (Clontech 631449) and 6 μg/ml polybrene (Sigma H9268) were added and the suspension centrifuged at 600 g at 32° C. for 1 hour. The cells were incubated at 37° C. for 6 hours before transferring into Matrigel. Following Cre Gesicle treatment, cells were grown in organoid media without puromycin.

For conditional Aqp5 KO in Aqp5-FLIP-Puro organoids, organoids were dissociated by TrypLE (Gibco 12604) and washed with 10% FBS in Advanced DMEM/F12 (Invitrogen 12634-028). Cre Recombinase Gesicles (Clontech 631449) and 6 μg/ml polybrene (Sigma H9268) were added and the suspension centrifuged at 600 g at 32° C. for 1 hour. The cells were incubated at 37° C. for 6 hours before transferring into Matrigel. Following Cre Gesicle treatment, cells were grown in organoid media without puromycin.

To measure cell proliferation in organoids, equal numbers of cells were seeded in 96-well plates within Matrigel, left to grow for 3 days, and incubated for 2 h with CellTiter AQueous One Solution Assay (Promega G3582). The absorbance at 490 nm was measured using a plate reader as a readout of cell proliferation. Proliferation was also measured using the Click-iT EdU Cell Proliferation Kit with Alexa Fluor 594 (Life Technologies C10339) following manufacturer's recommendations. Whole mount organoids were imaged on the Evos M5000 (Thermofisher).

### Cell Line Culture

All tissues were processed according to standard protocols. Briefly, fresh tissues were fixed in 4% paraformaldehyde at 4° C. overnight, dehydrated, and processed into paraffin blocks. 8 μm tissue sections collected on glass slides were deparaffinated and rehydrated. For Hematoxylin & Eosin (H&E), FFPE sections were stained with Richard Allan Haematoxylin, differentiated with acid alcohol (1% HCl in 70% alcohol), followed by Scott's blue and Eosin counterstaining. For immunohistochemistry (IHC) and immunofluorescence (IF), antigen retrieval was performed on rehydrated slides in a citrate pH 6.1 (Dako, S169984) or pH 9.0 (Dako S236784) target retrieval solution at 121° C. in a pressure cooker. Primary antibodies used were rabbit anti-AQP5 (Life Technologies PA564195), mouse anti-E-cadherin (1:200, BD Biosciences 610181), rabbit anti-KI67 (1:200, Thermofisher MA5-14520), mouse anti-RFP (1:200, Abcam 129244), rabbit anti-Vimentin (1:500, Abcam ab92547). Secondary antibodies used were mouse or rabbit EnVision+ (DAKO) for IHC and anti-mouse or antirabbit Alexa Fluor 488, 568, or 647 (1:500, Invitrogen) for IF.

RNA Isolation and qPCR

Cell lysis for RNA extraction was performed using RLT Plus buffer (Qiagen) with bmercaptoethanol. RNA was purified from the cell extracts using the RNeasy Mini or Micro Kit (Qiagen) and used for cDNA synthesis with the Superscript III kit (Life Technologies), following manufacturer's instructions. qPCR was performed in triplicates using the GoTaq qPCR Master Mix (Promega A6002). qPCR reactions were run on the QuantStudio 7 Flex Real-Time PCR system (Applied Biosystems) and analysed using the double delta Ct method. For qPCR validation of RNA sequencing targets, cDNA amplification was performed using the Ovation Pico WTA System (NuGen 3302-60) following manufacturer's instructions in order to generate sufficient material for validation of a large number of targets. All qPCR primer sequences used are summarised in Tables 2 and 4.

### Transcriptome Profiling

For bulk RNA-sequencing, cells were collected directly in RLT Plus buffer (Qiagen) with bmercaptoethanol during FACS sorting. Total RNA was extracted using the RNeasy Micro Kit (Qiagen). The RNA integrity of all samples was verified by Agilent RNA 6000 Pico Chips (Agilent 5067-1513) and ran on the Agilent 2100 Bioanalyzer prior to proceeding with downstream library preparation methods. For Aqp5+ samples, qPCR was also performed to confirm enrichment of Aqp5 levels over the corresponding sorted Aqp5− samples. Due to the low amount of material available from sorted human cells, RNA from human samples was first amplified using the SMARTer Ultra Low RNA kit (Clontech 634936) prior to library construction and sequencing. For library construction, mRNA was enriched using oligo (dT) beads and double-stranded cDNA library generated following manufacturer's instructions. Sequencing of libraries was performed on the NovaSeq PE150 (Illumina) and Illumina realtime analysis software used for base-calling to obtain FASTQ files.

### Histology and Staining Methods

All tissues were processed according to standard protocols. Briefly, fresh tissues were fixed in 4% paraformaldehyde at 4° C. overnight, dehydrated, and processed into paraffin blocks. 8 μm tissue sections collected on glass slides were deparaffinated and rehydrated. For Hematoxylin & Eosin (H&E), formalin-fixed paraffin-embedded (FFPE) sections were stained with Richard Allan Haematoxylin, differentiated with acid alcohol (1% HCl in 70% alcohol), followed by Scott's blue and Eosin counterstaining. For immunohistochemistry (IHC) and immunofluorescence (IF), antigen retrieval was performed on rehydrated slides in a citrate pH 6.1 (Dako, S169984) or pH 9.0 (Dako S236784) target retrieval solution at 121° C. in a pressure cooker. Primary antibodies used were rabbit anti-AQP5 (Life Technologies PA564195), mouse anti-CHGA (1:200, Abcam 15160), mouse anti-E-cadherin (1:200, BD Biosciences 610181), rabbit anti-KI67 (1:200, Thermofisher MA5-14520), mouse anti-MUC5AC (1:200, Leica Biosystems NCL-HGM-45-M1), mouse anti-RFP (1:200, Abcam 129244), anti-TFF2, rabbit anti-Vimentin (1:500, Abcam ab92547). Secondary antibodies used were mouse or rabbit EnVision+ (DAKO) for IHC and anti-mouse or anti-rabbit Alexa Fluor 488, 568, or 647 (1:500, Invitrogen) for IF.

For in-situ hybridization (ISH) experiments, tissues were collected under RNase-free conditions and fixed in 4% paraformaldehyde at room temperature for 16 to 24 hours. RNAscope was performed using the RNAscope 2.5 High Definition Brown Assay (ACDbio 322300) and 2.5 High Definition Duplex Reagent Assay following manufacturer's instructions. The following mouse probes were used: Mm-Aqp5 (ACDbio 430021), Mm-Lgr5 (ACDbio 312171), Mm-Rgs5 (ACDbio 430181), Mm-Pthlh (ACDbio 456521), Mm-Hey1 (ACDbio 319021), Mm-Cd44 (ACDbio 476201), Mm-Cxcr4 (ACDbio 425901), Positive Control probe Ms PPIB (ACDbio 313911), and Negative Control probe DapB (ACDbio 310043). The following human probes were used: Hs-CLDN2 (ACDbio 492051), Hs-HEY2 (ACDbio 441761), Hs-PLA1A (ACDbio 536951), Hs-DCHS2 (ACDbio 1309261) and Hs-AQP5 (ACDbio 452371).

### Microscopy

For image acquisition, H&E and IHC slides were captured using the Nikon Ni-E microscope with DS-Ri2 camera. IF images were acquired using the Zeiss LSM780 laser scanning confocal microscope. Brightfield and fluorescence images of organoids were taken with the Thermofisher Evos M5000 system. Large-area images acquired on the Nikon Ni-E microscope were processed with NIS-Elements AR software (Nikon), and those acquired on the Thermofisher Evos M5000 microscope were stitched using Adobe Photoshop. All acquired images were processed using Fiji and Adobe Photoshop.

### Statistical Analyses

All statistical analyses were performed in GraphPad Prism. Qualitative datasets were analysed using the Fisher's exact test. Quantitative datasets were first evaluated for normality using the Shapiro-Wilk test. Variables that follow a normal distribution were analysed by the unpaired, two-tailed Student's t-test for two groups or ANOVA with Dunnett's multiple comparisons test for more than two groups, whereas variables that do not follow a normal distribution were analysed using an unpaired, two-tailed Mann-Whitney U-test for two groups or Kruskal-Wallis test with Dunn's multiple comparisons test for more than two groups. Reproducibility was confirmed by at least three independent experiments.

### Tables

TABLE 1
Top upregulated genes in mouse Aqp5+ pyloric tumour
cells compared to Aqp5− cells.

| | | Fold |
|---|---|---|
| Gene | | change |
| symbol | Gene name | (Log₂) |
| Comp | Cartilage oligomeric matrix protein | 5.050 |
| Enpp2 | Ectonucleotide pyrophosphatase/ | 4.777 |
| | phosphodiesterase 2 | |
| Fam107a | Family with sequence similarity 107, | 4.565 |
| | member A | |
| Tafa2 | TAFA chemokine like family member 2 | 4.560 |
| Crlf1 | Cytokine receptor-like factor 1 | 4.557 |
| Egflam | EGF-like, fibronectin type III | 4.546 |
| | and laminin G domains | |
| Ntng1 | Netrin G1 | 4.452 |
| Meltf | Melanotransferrin | 4.402 |
| Samd3 | Sterile alpha motif domain containing | 4.390 |
| | protein 3 | |
| Bpifb1 | BPI fold containing family B, member 1 | 4.320 |
| Gm4951 | Predicted gene 4951 | 4.298 |
| Khdc1a | KH domain containing 1A | 4.262 |
| Rgs5 | Regulator of G-protein signaling 5 | 4.241 |
| Apod | Apolipoprotein D | 4.230 |
| Gm7298 | Predicted gene 7298 | 4.184 |
| Trf | Transferrin | 3.904 |
| Ckb | Creatine kinase, brain | 3.901 |
| Shc4 | SHC (Src homology 2 domain | 3.798 |
| | containing) family, member 4 | |
| Pthlh | Parathyroid hormone-like peptide | 3.795 |
| Obscn | Obscurin, cytoskeletal calmodulin and | 3.774 |
| | titin-interacting RhoGEF | |
| Igfbp2 | Insulin-like growth factor binding protein 2 | 3.744 |
| Prok1 | Prokineticin 1 | 3.715 |
| Diras2 | DIRAS family, GTP-binding RAS-like 2 | 3.700 |
| Edar | Ectodysplasin-A receptor | 3.654 |
| Fbln5 | Fibulin 5 | 3.637 |
| Adamtsl3 | ADAMTS-like 3 | 3.541 |
| Itgb8 | Integrin beta 8 | 3.526 |
| Cym | Chymosin | 3.520 |
| Fndc5 | Fibronectin type III domain | 3.510 |
| | containing protein 5 | |
| Kif26a | Kinesin family member 26A | 3.469 |
| Atp12a | ATPase, H+/K+ transporting, | 3.455 |
| | nongastric, alpha polypeptide | |
| Prss12 | Protease, serine 12 neurotrypsin (motopsin) | 3.411 |
| Col14a1 | Collagen, type XIV, alpha 1 | 3.305 |
| Chrdl1 | Chordin-like 1 | 3.300 |
| Cxcl15 | Chemokine (C-X-C motif) ligand 15 | 3.290 |
| Hey1 | Hairy/enhancer-of-split related | 3.256 |
| | with YRPW motif 1 | |
| Tmem59l | Transmembrane protein 59-like | 3.249 |
| Lmcd1 | LIM and cysteine-rich domains 1 | 3.143 |
| Asgr1 | Asialoglycoprotein receptor 1 | 3.078 |
| Dysf | Dysferlin | 3.065 |
| Faim2 | Fas apoptotic inhibitory molecule 2 | 3.060 |
| Chit1 | Chitinase 1 (chitotriosidase) | 3.047 |
| Clmp | CXADR-like membrane protein | 3.017 |
| Soga3 | SOGA family member 3 | 3.008 |
| Zic3 | Zinc finger protein of the cerebellum 3 | 2.967 |
| Acss3 | Acyl-CoA synthetase short-chain | 2.946 |
| | family member 3 | |
| Cyp1b1 | Cytochrome P450, family 1, subfamily b, | 2.909 |
| | polypeptide 1 | |
| Dnah5 | Dynein, axonemal, heavy chain 5 | 2.878 |
| Col11a2 | Collagen, type XI, alpha 2 | 2.869 |
| Vipr2 | Vasoactive intestinal peptide receptor 2 | 2.842 |
| Sec1415 | SEC14-like lipid binding 5 | 2.842 |
| Aqp5 | Aquaporin 5 | 2.838 |
| Stat4 | Signal transducer and activator | 2.822 |
| | of transcription 4 | |
| Cpne4 | Copine 4 | 2.817 |
| Ism2 | Isthmin 2 | 2.811 |
| Slc9a3 | Solute carrier family 9 (sodium/ | 2.795 |
| | hydrogen exchanger), member 3 | |
| Alpl | Alkaline phosphatase, liver/bone/kidney | 2.785 |
| Tmem28 | NALCN channel auxiliary factor 2 | 2.778 |
| A2m | Alpha-2-macroglobulin | 2.778 |
| Pcdh18 | Protocadherin 18 | 2.758 |
| Nid2 | Nidogen 2 | 2.753 |
| Serpinb9b | Serine (or cysteine) peptidase inhibitor, | 2.747 |
| | clade B, member 9b | |
| Ltbp4 | Latent transforming growth factor beta | 2.742 |
| | binding protein 4 | |
| Chl1 | Cell adhesion molecule L1-like | 2.741 |
| Ndnf | Neuron-derived neurotrophic factor | 2.731 |
| Trpv4 | Transient receptor potential cation | 2.718 |
| | channel, subfamily V, member 4 | |
| St8sia4 | ST8 alpha-N-acetyl-neuraminide alpha-2,8- | 2.710 |
| | sialyltransferase 4 | |
| Cd53 | CD53 antigen | 2.683 |
| Egr3 | Early growth response 3 | 2.679 |
| Gem | GTP binding protein (gene overexpressed | 2.668 |
| | in skeletal muscle) | |
| Mgam | Maltase-glucoamylase | 2.656 |
| Kcna2 | Potassium voltage-gated channel, | 2.655 |
| | shaker-related subfamily, member 2 | |
| Greb1 | Gene regulated by estrogen in breast | 2.635 |
| | cancer protein | |
| Hey2 | Hairy/enhancer-of-split related with | 2.632 |
| | YRPW motif 2 | |
| Cpxm2 | Carboxypeptidase X 2 (M14 family) | 2.629 |
| Susd5 | Sushi domain containing 5 | 2.617 |
| Psors1c2 | Psoriasis susceptibility 1 candidate | 2.600 |
| | 2 (human) | |
| Asb4 | Ankyrin repeat and SOCS box-containing 4 | 2.590 |
| Gad2 | Glutamic acid decarboxylase 2 | 2.576 |
| Igsf1 | Immunoglobulin superfamily, member 1 | 2.571 |
| Slco4c1 | Solute carrier organic anion transporter | 2.561 |
| | family, member 4C1 | |
| Gfra1 | Glial cell line derived neurotrophic | 2.551 |
| | factor family receptor alpha 1 | |
| Kcnq5 | Potassium voltage-gated channel, | 2.531 |
| | subfamily Q, member 5 | |
| Abcb1a | ATP-binding cassette, sub-family B | 2.524 |
| | (MDR/TAP), member 1A | |
| Cxcr4 | Chemokine (C-X-C motif) receptor 4 | 2.520 |
| Dpyd | Dihydropyrimidine dehydrogenase | 2.519 |
| Adamts1 | A disintegrin-like and | 2.513 |
| | metallopeptidase (reprolysin | |
| | type) with thrombospondin type 1 motif, 1 | |
| Gprc6a | G protein-coupled receptor, family C, | 2.509 |
| | group 6, member A | |
| Gnal | Guanine nucleotide binding protein, alpha | 2.482 |
| | stimulating, olfactory type | |
| Tnfrsf19 | Tumor necrosis factor receptor | 2.470 |
| | superfamily, member 19 | |
| Pcsk5 | Proprotein convertase subtilisin/ | 2.451 |
| | kexin type 5 | |
| Car12 | Carbonic anhydrase 12 | 2.444 |
| Rfx6 | Regulatory factor X, 6 | 2.440 |
| Fam171b | Family with sequence similarity | 2.438 |
| | 171, member B | |
| Snx22 | Sorting nexin 22 | 2.427 |
| Gpr3 | G-protein coupled receptor 3 | 2.421 |
| 9330159F19Rik | RIKEN cDNA 9330159F19 gene | 2.418 |
| Tigit | T cell immunoreceptor with Ig and | 2.413 |
| | ITIM domains | |
| Nptx2 | Neuronal pentraxin 2 | 2.408 |
| Col4a1 | Collagen, type IV, alpha 1 | 2.400 |
| Grm4 | Glutamate receptor, metabotropic 4 | 2.399 |
| Lepr | Leptin receptor | 2.389 |
| S1pr1 | Sphingosine-1-phosphate receptor 1 | 2.378 |
| Cttnbp2 | Cortactin binding protein 2 | 2.377 |
| Gng11 | Guanine nucleotide binding protein | 2.371 |
| | (G protein), gamma 11 | |
| Mug1 | Murinoglobulin 1 | 2.362 |
| Trpm1 | Transient receptor potential cation channel, | 2.359 |
| | subfamily M, member 1 | |
| Tcf24 | Transcription factor 24 | 2.335 |
| Rnf125 | Ring finger protein 125 | 2.329 |
| Fam184a | Family with sequence similarity | 2.320 |
| | 184, member A | |
| Dmrta1 | Doublesex and mab-3 related | 2.310 |
| | transcription factor like family A1 | |
| Drd3 | Dopamine receptor D3 | 2.310 |
| Sult4a1 | Sulfotransferase family 4A, member 1 | 2.302 |
| Plb1 | Phospholipase B1 | 2.299 |
| Scd3 | Stearoyl-coenzyme A desaturase 3 | 2.295 |
| Epp13 | Epididymal protein 13 | 2.288 |
| Wfdc18 | WAP four-disulfide core domain 18 | 2.283 |
| Tmtc1 | Transmembrane and tetratricopeptide repeat | 2.280 |
| | containing 1 | |
| Paqr6 | Progestin and adipoQ receptor | 2.279 |
| | family member VI | |
| Xkr4 | X-linked Kx blood group related 4 | 2.266 |
| Me3 | Malic enzyme 3, NADP(+)- | 2.264 |
| | dependent, mitochondrial | |
| Bpifb4 | BPI fold containing family B, member 4 | 2.259 |
| Pxdn | Peroxidasin | 2.248 |
| Afap1l1 | Actin filament associated protein 1-like 1 | 2.245 |
| Clip4 | CAP-GLY domain containing linker | 2.239 |
| | protein family, member 4 | |
| Nlrp10 | NLR family, pyrin domain containing 10 | 2.216 |
| Elf5 | E74-like factor 5 | 2.211 |
| Msx3 | Msh homeobox 3 | 2.208 |
| Rflna | Refilin A | 2.206 |
| Pla2g5 | Phospholipase A2, group V | 2.190 |
| Sdc3 | Syndecan 3 | 2.182 |
| Entpd3 | Ectonucleoside triphosphate | 2.165 |
| | diphosphohydrolase 3 | |
| Gprc5b | G protein-coupled receptor, family C, | 2.163 |
| | group 5, member B | |
| Plod2 | Procollagen lysine, 2-oxoglutarate | 2.159 |
| | 5-dioxygenase 2 | |
| Iigp1 | Interferon inducible GTPase 1 | 2.157 |
| Jaml | Junction adhesion molecule like | 2.154 |
| Apob | Apolipoprotein B | 2.150 |
| Serpinb9 | Serine (or cysteine) peptidase inhibitor, | 2.150 |
| | clade B, member 9 | |
| Rnf149 | Ring finger protein 149 | 2.146 |
| Ndp | Norrie disease (pseudoglioma) | 2.136 |
| Cubn | Cubilin (intrinsic factor-cobalamin receptor) | 2.133 |
| Fgfr4 | Fibroblast growth factor receptor 4 | 2.133 |
| P2ry1 | Purinergic receptor P2Y, G-protein | 2.127 |
| | coupled 1 | |
| Ggh | Gamma-glutamyl hydrolase | 2.123 |
| Klhl13 | Kelch-like 13 | 2.122 |
| Zbtb20 | Zinc finger and BTB domain containing 20 | 2.118 |
| Timp3 | Tissue inhibitor of metalloproteinase 3 | 2.110 |
| Sh2d5 | SH2 domain containing 5 | 2.108 |
| Sp5 | Trans-acting transcription factor 5 | 2.103 |
| Npy | Neuropeptide Y | 2.101 |
| Foxc1 | Forkhead box C1 | 2.097 |
| Tns4 | Tensin 4 | 2.090 |
| Col4a2 | Collagen, type IV, alpha 2 | 2.074 |
| Serpina3n | Serine (or cysteine) peptidase inhibitor, | 2.066 |
| | clade A, member 3N | |
| Tmod1 | Tropomodulin 1 | 2.063 |
| Pde8b | Phosphodiesterase 8B | 2.061 |
| Atp6v1b1 | ATPase, H+ transporting, lysosomal | 2.047 |
| | V1 subunit B1 | |
| Dact2 | Dishevelled-binding antagonist | 2.045 |
| | of beta-catenin 2 | |
| Ccnjl | Cyclin J-like | 2.027 |
| Serpinb6b | Serine (or cysteine) peptidase inhibitor, | 2.026 |
| | clade B, member 6b | |
| Rundc3b | RUN domain containing 3B | 2.019 |
| Cxcl10 | Chemokine (C-X-C motif) ligand 10 | 2.013 |
| Cldn34c1 | Claudin 34C1 | 2.012 |
| Trps1 | Transcriptional repressor GATA binding 1 | 2.010 |
| C1qtnf1 | C1q and tumor necrosis factor | 2.008 |
| | related protein 1 | |
| Nckap5 | NCK-associated protein 5 | 2.000 |

TABLE 2
Mouse target validation qPCR primer sequences.

| Gene | Forward primer | Reverse primer |
|---|---|---|
| mAbcb1a | TCCTCACCAAGCGACTCCGATA | ACTTGAGCAGCATCGTTGGCGA |
| | (SEQ ID NO: 1) | (SEQ ID NO: 2) |
| mAcss3 | CAGAGTTCTCATTTACAGCCGTC | AGAGAACAGGCACGCAGTCATG |
| | (SEQ ID NO: 3) | (SEQ ID NO: 4) |
| mAdamtsl3 | ATCCGCTTGAAGAGGGTGGT | AGGCTGGAAGTGGTCATAGGG |
| | (SEQ ID NO: 5) | (SEQ ID NO: 6) |
| mAqp5 | TCCATGAACCCAGCCCGATCTT | GAAGTAGAGGATTGCAGCCAGG |
| | (SEQ ID NO: 7) | (SEQ ID NO: 8) |
| mAsgr1 | TGAGCACTGAGGACCAGGTCAA | CGTCAGACACTAACTGCTTCACG |
| | (SEQ ID NO: 9) | (SEQ ID NO: 10) |
| mClmp | CCTGGACATTGAATGGCTGCTC | GAAGCATCTCCTGCCAGGAAGT |
| | (SEQ ID NO: 11) | (SEQ ID NO: 12) |
| mCyb1p1 | GCCACTATTACGGACATCTTCGG | ACAACCTGGTCCAACTCAGCCT |
| | (SEQ ID NO: 13) | (SEQ ID NO: 14) |
| mGapdh | CATCACTGCCACCCAGAAGACTG | ATGCCAGTGAGCTTCCCGTTCAG |
| | (SEQ ID NO: 15) | (SEQ ID NO: 16) |
| mHey1 | CCAACGACATCGTCCCAGGTTT | CTGCTTCTCAAAGGCACTGGGT |
| | (SEQ ID NO: 17) | (SEQ ID NO: 18) |
| mltgb8 | ATGCTTCAGGCTGCCGTCTGTG | CAGTTTCCGTCATTCGGCACCA |
| | (SEQ ID NO: 19) | (SEQ ID NO: 20) |
| mMeltf | CCTGTCGGTTACCTCGTAGAGA | GGACTCGGAATGGCTGGTTTCT |
| | (SEQ ID NO: 21) | (SEQ ID NO: 22) |
| mPthlh | GGCGTTCGGTGGAGGGGCTT | CAGATGGTGGAGGAAGAAACGG |
| | (SEQ ID NO: 23) | (SEQ ID NO: 24) |
| mRgs5 | CAAAATGGCGGAGAAGGCAAAGC | TCAAAGCTGCGAGGAGACGGTT |
| | (SEQ ID NO: 25) | (SEQ ID NO: 26) |
| mShc4 | GCAATAAGTCGCCTGTGTGAAGC | CATCAGTGTGAGGCTGCTTGTTG |
| | (SEQ ID NO: 27) | (SEQ ID NO: 28) |

TABLE 3
Top upregulated genes in human Aqp5+ pyloric tumour cells
compared to Aqp5− cells.

| | | Fold |
|---|---|---|
| | | change |
| Gene symbol | Gene name | (Log₂) |
| Atp4a | ATPase H+/K+ transporting subunit alpha | 12.582 |
| Itga8 | Integrin subunit alpha 8 | 9.375 |
| Sgcd | Sarcoglycan delta | 9.063 |
| Sgca | Sarcoglycan alpha | 9.034 |
| Ackr1 | Atypical chemokine receptor 1 | 9.028 |
| Pla1a | Phospholipase A1 member A | 8.648 |
| Ntrk2 | Neurotrophic receptor tyrosine kinase 2 | 8.593 |
| Ccl18 | C-C motif chemokine ligand 18 | 8.445 |
| Clec14a | C-type lectin domain containing 14A | 8.239 |
| Folr2 | Folate receptor beta | 8.092 |
| Ca4 | Carbonic anhydrase 4 | 7.944 |
| Col23a1 | Collagen type XXIII alpha 1 chain | 7.895 |
| Kcnj5 | Potassium inwardly rectifying channel | 7.832 |
| | subfamily J member 5 | |
| Ldb2 | LIM domain binding 2 | 7.701 |
| Cd163l1 | CD163 molecule like 1 | 7.430 |
| II2ra | Interleukin 2 receptor subunit alpha | 7.392 |
| Hey2 | Hes related family bHLH transcription | 7.346 |
| | factor with YRPW motif 2 | |
| Olfml2a | Olfactomedin like 2A | 7.149 |
| Bag2 | BAG cochaperone 2 | 7.119 |
| Colec11 | Collectin subfamily member 11 | 7.069 |
| Ms4a4a | Membrane spanning 4-domains A4A | 6.881 |
| Pde4b | Phosphodiesterase 4B | 6.878 |
| Ip6k3 | Inositol hexakisphosphate kinase 3 | 6.737 |
| Cdh6 | Cadherin 6 | 6.720 |
| Prkaa2 | Protein kinase AMP-activated | 6.569 |
| | catalytic subunit alpha 2 | |
| Moxd1 | Monooxygenase DBH like 1 | 6.543 |
| Cdh5 | Cadherin 5 | 6.498 |
| Kcna5 | Potassium voltage-gated channel | 6.470 |
| | subfamily A member 5 | |
| Heyl | Hes related family bHLH transcription | 6.300 |
| | factor with YRPW motif like | |
| Adgrl4 | Adhesion G protein-coupled receptor L4 | 6.278 |
| Glb1l3 | Galactosidase beta 1 like 3 | 6.271 |
| Kcnj8 | Potassium inwardly rectifying | 6.260 |
| | channel subfamily J member 8 | |
| Scgb3a1 | Secretoglobin family 3A member 1 | 6.229 |
| Sulf1 | Sulfatase 1 | 6.178 |
| Art3 | ADP-ribosyltransferase 3 | 6.090 |
| Rnls | Renalase, FAD dependent amine oxidase | 5.960 |
| Gucy1b3 | Guanylate cyclase 1 soluble subunit beta 1 | 5.954 |
| Nell1 | Neural EGFL like 1 | 5.917 |
| Ncam1 | Neural cell adhesion molecule 1 | 5.897 |
| Clul1 | Clusterin like 1 | 5.818 |
| II4i1 | Interleukin 4 induced 1 | 5.816 |
| Fgr | FGR proto-oncogene, Src family | 5.768 |
| | tyrosine kinase | |
| Aoc3 | Amine oxidase copper containing 3 | 5.600 |
| Gucy1a3 | Guanylate cyclase 1 soluble subunit alpha 1 | 5.507 |
| Slc1a2 | Solute carrier family 1 member 2 | 5.365 |
| Bgn | Biglycan | 5.228 |
| Cnn1 | Calponin 1 | 5.177 |
| Plvap | Plasmalemma vesicle associated protein | 5.142 |
| Itga11 | Integrin subunit alpha 11 | 5.135 |
| Palmd | Palmdelphin | 5.086 |
| Gng11 | G protein subunit gamma 11 | 4.846 |
| Lipf | Lipase F, gastric type | 4.818 |
| Colec12 | Collectin subfamily member 12 | 4.769 |
| C3orf18 | Chromosome 3 open reading frame 18 | 4.727 |
| Mfap5 | Microfibril associated protein 5 | 4.669 |
| Myrip | Myosin VIIA and Rab interacting protein | 4.656 |
| Stk32b | Serine/threonine kinase 32B | 4.598 |
| Trpv4 | Transient receptor potential cation | 4.451 |
| | channel subfamily V member 4 | |
| Shisa6 | Shisa family member 6 | 4.438 |
| Ackr3 | Atypical chemokine receptor 3 | 4.431 |
| Clmp | CXADR like membrane protein | 4.420 |
| Igfals | Insulin like growth factor binding | 4.380 |
| | protein acid labile subunit | |
| Pln | Phospholamban | 4.313 |
| Arhgap40 | Rho GTPase activating protein 40 | 4.265 |
| Padi2 | Peptidyl arginine deiminase 2 | 4.247 |
| Wnt5a | Wnt family member 5A | 4.224 |
| Tnnc1 | Troponin C1, slow skeletal and | 4.079 |
| | cardiac type | |
| Ptn | Pleiotrophin | 4.064 |
| Fcgr2a | Fc fragment of IgG receptor lia | 4.062 |
| Mcam | Melanoma cell adhesion molecule | 4.060 |
| Rnase6 | Ribonuclease A family member k6 | 3.919 |
| C6orf58 | Chromosome 6 open reading frame 58 | 3.917 |
| Serpina5 | Serpin family A member 5 | 3.917 |
| Msrb3 | Methionine sulfoxide reductase B3 | 3.867 |
| Vsig4 | V-set and immunoglobulin | 3.827 |
| | domain containing 4 | |
| Notum | Notum, palmitoleoyl-protein | 3.813 |
| | carboxylesterase | |
| Lrrc2 | Leucine rich repeat containing 2 | 3.732 |
| Acta2 | Actin alpha 2, smooth muscle | 3.721 |
| Cdh11 | Cadherin 11 | 3.719 |
| Col11a2 | Collagen type X1 alpha 2 chain | 3.704 |
| Cldn10 | Claudin 10 | 3.670 |
| Gnb4 | G protein subunit beta 4 | 3.660 |
| Acp5 | Acid phosphatase 5, tartrate resistant | 3.611 |
| Cldn2 | Claudin 2 | 3.584 |
| Cckbr | Cholecystokinin B receptor | 3.581 |
| Dchs2 | Dachsous cadherin-related 2 | 3.578 |
| Myh11 | Myosin heavy chain 11 | 3.543 |
| Cryab | Crystallin alpha B | 3.534 |
| C1qa | Complement C1q A chain | 3.513 |
| Muc6 | Mucin 6, oligomeric mucus/gel-forming | 3.512 |
| Vcan | Versican | 3.399 |
| Odam | Odontogenic, ameloblast associated | 3.308 |
| Mcoln2 | Mucolipin 2 | 3.267 |
| Irx3 | Iroquois homeobox 3 | 3.258 |
| Colgalt2 | Collagen beta(1-O)galactosyltransferase 2 | 3.257 |
| Tnfrsf19 | TNF receptor superfamily member 19 | 3.256 |
| Vasn | Vasorin | 3.236 |
| Htra3 | HtrA serine peptidase 3 | 3.235 |
| Actg2 | Actin gamma 2, smooth muscle | 3.213 |
| Slit3 | Slit guidance ligand 3 | 3.213 |
| Rcan2 | Regulator of calcineurin 2 | 3.198 |
| Ltf | Lactotransferrin | 3.188 |
| P3h3 | Prolyl 3-hydroxylase 3 | 3.131 |
| Hla-dqa1 | Major histocompatibility complex, | 3.123 |
| | class II, DQ alpha 1 | |
| Scara3 | Scavenger receptor class A member 3 | 3.119 |
| Aqp5 | Aquaporin 5 | 3.069 |
| A4gnt | Alpha-1,4-N- | 3.055 |
| | acetylglucosaminyltransferase | |
| Fmod | Fibromodulin | 3.048 |
| Tubb6 | Tubulin beta 6 class V | 3.010 |
| Apold1 | Apolipoprotein L domain containing 1 | 2.977 |
| Tpm2 | Tropomyosin 2 | 2.975 |
| Notch3 | Notch receptor 3 | 2.959 |
| Chrm3 | Cholinergic receptor muscarinic 3 | 2.957 |
| Lmcd1 | LIM and cysteine rich domains 1 | 2.953 |
| Wfdc1 | WAP four-disulfide core domain 1 | 2.951 |
| C16orf89 | Chromosome 16 open reading frame 89 | 2.906 |
| Myl9 | Myosin light chain 9 | 2.844 |
| Sparcl1 | SPARC like 1 | 2.837 |
| Prr4 | Proline rich 4 | 2.830 |
| Fut1 | Fucosyltransferase 1 (H blood group) | 2.796 |
| C17orf78 | Chromosome 17 open reading frame 78 | 2.734 |
| Cercam | Cerebral endothelial cell adhesion molecule | 2.713 |
| Gp2 | Glycoprotein 2 | 2.713 |
| Ldoc1 | LDOC1 regulator of NFKB signaling | 2.689 |
| Krt17 | Keratin 17 | 2.689 |
| Prss21 | Serine protease 21 | 2.682 |
| Bpifb1 | BPI fold containing family B member 1 | 2.679 |
| Csgalnact1 | Chondroitin sulfate N- | 2.660 |
| | acetylgalactosaminyltransferase 1 | |
| C10orf11 | Leucine rich melanocyte | 2.658 |
| | differentiation associated | |
| Gyltl1b | LARGE xylosyl- and | 2.651 |
| | glucuronyltransferase 2 | |
| Tns1 | Tensin 1 | 2.643 |
| Nexn | Nexilin F-actin binding protein | 2.643 |
| Hpn | Hepsin | 2.640 |
| Gmpr | Guanosine monophosphate reductase | 2.635 |
| Anxa6 | Annexin A6 | 2.626 |
| Sntb1 | Syntrophin beta 1 | 2.624 |
| Tmem45a | Transmembrane protein 45A | 2.611 |
| Fermt2 | FERM domain containing kindlin 2 | 2.602 |
| Igfbp7 | Insulin like growth factor binding | 2.596 |
| | protein 7 | |
| Cecr1 | Adenosine deaminase 2 | 2.576 |
| Reg1a | Regenerating family member 1 alpha | 2.525 |
| Serpina4 | Serpin family A member 4 | 2.449 |
| Dcbld2 | Discoidin, CUB and LCCL | 2.431 |
| | domain containing 2 | |
| Nnmt | Nicotinamide N-methyltransferase | 2.430 |
| Col18a1 | Collagen type XVIII alpha 1 chain | 2.415 |
| Pgc | Progastricsin | 2.411 |
| Habp2 | Hyaluronan binding protein 2 | 2.405 |
| Acsm3 | Acyl-CoA synthetase medium | 2.374 |
| | chain family member 3 | |
| Cald1 | Caldesmon 1 | 2.330 |
| Fads2 | Fatty acid desaturase 2 | 2.327 |
| Sod3 | Superoxide dismutase | 2.285 |
| Vim | Vimentin | 2.268 |
| Rp3-412a9.11 | | 2.268 |
| Adamts1 | ADAM metallopeptidase with | 2.265 |
| | thrombospondin type 1 motif 1 | |
| Kcne2 | Potassium voltage-gated channel | 2.245 |
| | subfamily E regulatory subunit 2 | |
| Rgs5 | Regulator of G protein signaling 5 | 2.233 |
| Kcns3 | Potassium voltage-gated channel | 2.206 |
| | modifier subfamily S member 3 | |
| Reg3a | Regenerating family member 3 alpha | 2.201 |
| Sparc | Secreted protein acidic and cysteine rich | 2.197 |
| Slc39a8 | Solute carrier family 39 member 8 | 2.197 |
| Znf362 | Zinc finger protein 362 | 2.182 |
| Slc2a3 | Solute carrier family 2 member 3 | 2.166 |
| Rarres2 | Retinoic acid receptor responder 2 | 2.165 |
| Etv5 | ETS variant transcription factor 5 | 2.163 |
| Cadm1 | Cell adhesion molecule 1 | 2.156 |
| Frzb | Frizzled related protein | 2.149 |
| Prss23 | Serine protease 23 | 2.142 |
| Thy1 | Thy-1 cell surface antigen | 2.127 |
| Ctsl | Cathepsin L | 2.121 |
| Ca12 | Carbonic anhydrase 12 | 2.118 |
| Tspan4 | Tetraspanin 4 | 2.088 |
| Tcn1 | Transcobalamin 1 | 2.085 |
| Pmepa1 | Prostate transmembrane protein, | 2.070 |
| | androgen induced 1 | |
| Mmp7 | Matrix metallopeptidase 7 | 2.056 |
| Serpina1 | Serpin family A member 1 | 2.036 |
| Slc22a17 | Solute carrier family 22 member 17 | 2.030 |
| Pltp | Phospholipid transfer protein | 2.017 |
| Thbs 1 | Thrombospondin 1 | 2.012 |
| Csrp2 | Cysteine and glycine rich protein 2 | 2.008 |
| Ctsf | Cathepsin F | 2.006 |
| Creb3l4 | CAMP responsive element binding | 2.005 |
| | protein 3 like 4 | |
| Efemp1 | EGF containing fibulin extracellular | 2.000 |
| | matrix protein 1 | |

TABLE 4
Human target validation qPCR primer sequences.

| Gene | Forward primer | Reverse primer |
|---|---|---|
| hAqp5 | TACGGTGTGGCACCGCTCAATG | AGTCAGTGGAGGCGAAGATGCA |
| | (SEQ ID NO: 29) | (SEQ ID NO: 30) |
| hBag2 | TAGCCAGGACATGAGGCAGATC | GCTTTAGGGATTCTTGCTGCTGG |
| | (SEQ ID NO: 31) | (SEQ ID NO: 32) |
| hCldn2 | GTGACAGCAGTTGGCTTCTCCA | GGAGATTGCACTGGATGTCACC |
| | (SEQ ID NO: 33) | (SEQ ID NO: 34) |
| hDchs2 | CTGGAGAGGTAGTGACAACCAC | GCAGAGGATTGTTGTGGTGCTG |
| | (SEQ ID NO: 35) | (SEQ ID NO: 36) |
| hGapdh | GTCTCCTCTGACTTCAACAGCG | ACCACCCTGTTGCTGTAGCCAA |
| | (SEQ ID NO: 37) | (SEQ ID NO: 38) |
| hHey2 | TGAGAAGACTTGTGCCAACTGCT | CCCTGTTGCCTGAAGCATCTTC |
| | (SEQ ID NO: 39) | (SEQ ID NO: 40) |
| hMfap5 | GGGTCAATAGTCAACGAGGAGAC | GCCAAGTCATCTGTGGAAGGTG |
| | (SEQ ID NO: 41) | (SEQ ID NO: 42) |
| hMsrb3 | CTATGGGATGCACAGGGTGGAA | CGCAGGTGTAAAAGACAAGGCAG |
| | (SEQ ID NO: 43) | (SEQ ID NO: 44) |
| hPla1a | ACCTCCTGACTACTTCCAGTGC | GGAAGGTAACCTCGATGTTGGTG |
| | (SEQ ID NO: 45) | (SEQ ID NO: 46) |
| hVsig4 | GATGGCAACCAAGTCGTGAGAG | CCTGGCATTGAAGGCTAATCCTC |
| | (SEQ ID NO: 47) | (SEQ ID NO: 48) |

SEQUENCE LISTING

| SEQ ID NO: | Name | Sequence |
|---|---|---|
| 1 | Mouse Abcb1a-forward | TCCTCACCAAGCGACTCCGATA |
| | primer | |
| 2 | Mouse Abcb1a-reverse | ACTTGAGCAGCATCGTTGGCGA |
| | primer | |
| 3 | Mouse Acss3-forward | CAGAGTTCTCATTTACAGCCGTC |
| | primer | |
| 4 | Mouse Acss3-reverse | AGAGAACAGGCACGCAGTCATG |
| | primer | |
| 5 | Mouse Adamtsl3- | ATCCGCTTGAAGAGGGTGGT |
| | forward primer | |
| 6 | Mouse Adamtsl3- | AGGCTGGAAGTGGTCATAGGG |
| | reverse primer | |
| 7 | Mouse Aqp5-forward | TCCATGAACCCAGCCCGATCTT |
| | primer | |
| 8 | Mouse Aqp5-reverse | GAAGTAGAGGATTGCAGCCAGG |
| | primer | |
| 9 | Mouse Asgr1-forward | TGAGCACTGAGGACCAGGTCAA |
| | primer | |
| 10 | Mouse Asgr1-reverse | CGTCAGACACTAACTGCTTCACG |
| | primer | |
| 11 | Mouse Clmp-forward | CCTGGACATTGAATGGCTGCTC |
| | primer | |
| 12 | Mouse Clmp-reverse | GAAGCATCTCCTGCCAGGAAGT |
| | primer | |
| 13 | Mouse Cyb1p1-forward | GCCACTATTACGGACATCTTCGG |
| | primer | |
| 14 | Mouse Cyb1p1-reverse | ACAACCTGGTCCAACTCAGCCT |
| | primer | |
| 15 | Mouse Gapdh-forward | CATCACTGCCACCCAGAAGACTG |
| | primer | |
| 16 | Mouse Gapdh-reverse | ATGCCAGTGAGCTTCCCGTTCAG |
| | primer | |
| 17 | Mouse Hey1-forward | CCAACGACATCGTCCCAGGTTT |
| | primer | |
| 18 | Mouse Hey1-reverse | CTGCTTCTCAAAGGCACTGGGT |
| | primer | |
| 19 | Mouse Itgb8-forward | ATGCTTCAGGCTGCCGTCTGTG |
| | primer | |
| 20 | Mouse Itgb8-reverse | CAGTTTCCGTCATTCGGCACCA |
| | primer | |
| 21 | Mouse Meltf-forward | CCTGTCGGTTACCTCGTAGAGA |
| | primer | |
| 22 | Mouse Meltf-reverse | GGACTCGGAATGGCTGGTTTCT |
| | primer | |
| 23 | Mouse Pthlh-forward | GGCGTTCGGTGGAGGGGCTT |
| | primer | |
| 24 | Mouse Pthlh-reverse | CAGATGGTGGAGGAAGAAACGG |
| | primer | |
| 25 | Mouse Rgs5-forward | CAAAATGGCGGAGAAGGCAAAGC |
| | primer | |
| 26 | Mouse Rgs5-reverse | TCAAAGCTGCGAGGAGACGGTT |
| | primer | |
| 27 | Mouse Shc4-forward | GCAATAAGTCGCCTGTGTGAAGC |
| | primer | |
| 28 | Mouse Shc4-reverse | CATCAGTGTGAGGCTGCTTGTTG |
| | primer | |
| 29 | Human Aqp5-forward | TACGGTGTGGCACCGCTCAATG |
| | primer | |
| 30 | Human Aqp5-reverse | AGTCAGTGGAGGCGAAGATGCA |
| | primer | |
| 31 | Human Bag2-forward | TAGCCAGGACATGAGGCAGATC |
| | primer | |
| 32 | Human Bag2-reverse | GCTTTAGGGATTCTTGCTGCTGG |
| | primer | |
| 33 | Human Cldn2-forward | GTGACAGCAGTTGGCTTCTCCA |
| | primer | |
| 34 | Human Cldn2-reverse | GGAGATTGCACTGGATGTCACC |
| | primer | |
| 35 | Human Dchs2-forward | CTGGAGAGGTAGTGACAACCAC |
| | primer | |
| 36 | Human Dchs2-reverse | GCAGAGGATTGTTGTGGTGCTG |
| | primer | |
| 37 | Human Gapdh-forward | GTCTCCTCTGACTTCAACAGCG |
| | primer | |
| 38 | Human Gapdh-reverse | ACCACCCTGTTGCTGTAGCCAA |
| | primer | |
| 39 | Human Hey2-forward | TGAGAAGACTTGTGCCAACTGCT |
| | primer | |
| 40 | Human Hey2-reverse | CCCTGTTGCCTGAAGCATCTTC |
| | primer | |
| 41 | Human Mfap5-forward | GGGTCAATAGTCAACGAGGAGAC |
| | primer | |
| 42 | Human Mfap5-reverse | GCCAAGTCATCTGTGGAAGGTG |
| | primer | |
| 43 | Human Msrb3-forward | CTATGGGATGCACAGGGTGGAA |
| | primer | |
| 44 | Human Msrb3-reverse | CGCAGGTGTAAAAGACAAGGCAG |
| | primer | |
| 45 | Human Pla1a-forward | ACCTCCTGACTACTTCCAGTGC |
| | primer | |
| 46 | Human Pla1a-reverse | GGAAGGTAACCTCGATGTTGGTG |
| | primer | |
| 47 | Human Vsig4-forward | GATGGCAACCAAGTCGTGAGAG |
| | primer | |
| 48 | Human Vsig4-reverse | CCTGGCATTGAAGGCTAATCCTC |
| | primer | |
| 49 | siRNA primer pair 1- | 5′-AAAACTCTGCGAACACGGCCCCTGTCTC-3′ |
| | sense | |
| 50 | siRNA primer pair 1- | 5′-AAGGCCGTGTTCGCAGAGTTCCTGTCTC-3′ |
| | antisense | |
| 51 | siRNA primer pair 2- | 5′-CGGUGGUCAUGAAUCGGUUTT-3′ |
| | sense | |
| 52 | siRNA primer pair 2- | 5′-AACCGAUUCAUGACCACCGCA-3′ |
| | antisense | |
| 53 | siRNA primer pair 3- | 5′-GCGUGUGGCCAUCAUCAAATT-3′ |
| | sense | |
| 54 | siRNA primer pair 3- | 5′-UUUGAUGAUGGCCACACGCTT-3′ |
| | antisense | |

1. A method of identifying a gastric cancer stem cell or gastric cancer stem cell population comprising:
a1) detecting expression of Aquaporin 5 (AQP5) in a cell or cell population; or
b1) detecting expression level of AQP5 in a cell or a cell population and comparing the expression level with the expression level of AQP5 in a reference cell or reference cell population,
wherein detection of AQP5 expression in the cell or cell population, or expression of AQP5 in the cell or cell population at an increased level compared to the reference cell or reference cell population identifies said cell or cell population as a gastric cancer stem cell or gastric cancer stem cell population.

2. The method of claim 1, wherein the cell or cell population is an in vitro, in vivo, ex vivo cell or cell population; or wherein the cell or cell population is a gastric tumor sample, biopsy, or organoid.

3.-4. (canceled)

5. The method of claim 1, wherein the cell or cell population is modified to express AQP5 in conjunction with one or more detectable labels; and/or wherein the cell or cell population is further modified to express AQP5 in conjunction with an inducible gene.

6.-8. (canceled)

9. The method of claim 1, wherein the cell or cell population is modified to express AQP5 in conjunction with a diphtheria toxin receptor (DTR) gene or an inducible Caspase9 (iCasp9) gene.

10. The method of claim 1, wherein the expression level of AQP5 is gene expression level, protein expression level, or a combination thereof.

11. The method of claim 1, further comprising isolating the identified gastric cancer stem cell.

12. (canceled)

13. The method of claim 11, wherein the isolated gastric cancer stem cell forms an AQP5-expressing tumor organoid.

14. The method of claim 1, further comprising eliminating or ablating the gastric cancer stem cell.

15. (canceled)

16. The method of claim 1, wherein the reference cell or cell population is a cell that does not express AQP5, optionally wherein the cell that does not express AQP5 is a non-gastric cell, a non-cancerous gastric cell, or combinations thereof.

17. The method of claim 1, wherein identification of a gastric cancer stem cell results in the subject being treated with a standard of care treatment for gastric cancer.

18. A method of isolating one or more gastric cancer stem cells from a cell population, comprising:
i) contacting cells of the cell population with an agent that binds to AQP5;
ii) isolating one or more AQP5-expressing cells that are bound to the agent, wherein the one or more AQP5-expressing cells are gastric cancer stem cells.

19. The method of claim 18 wherein the agent that binds to AQP5 is an antibody.

20. The method of claim 19, wherein the antibody is conjugated to a label, such as a detectable label.

21. The method of claim 18, wherein the one or more gastric cancer stem cells are isolated using a method selected from the group consisting of single cell sorting, fluorescent activated cell sorting, and magnetic sorting.

22. The method of claim 18, wherein the isolated gastric cancer stem cell is further cultured in the presence of culture media that does not comprise growth factors.

23. The method of claim 22, wherein the isolated gastric cancer stem cell forms an AQP5-expressing tumor organoid.

24.-25. (canceled)

26. A method of ablating or eliminating an AQP5+ gastric cancer stem cell, wherein a) the AQP5+ gastric cancer stem cell has been modified to express the DTR gene or an inducible Caspase9 (iCasp9) gene, comprising contacting said cell with a diphtheria toxin (DT) or an inducer of iCasp9; or b) the method comprising contacting the cell with an agent that binds to AQP5, wherein binding of the AQP5+ cell with the agent ablates or eliminates the cell.

27.-28. (canceled)

29. The method of claim 1, wherein AQP5 is membrane-bound.

30. A method of treating gastric cancer in a subject in need thereof, comprising administering a therapeutically effective amount of one or more agents that eliminates or ablates AQP5-expressing cells to the subject.

31. (canceled)

32. A biomarker of gastric cancer stem cells, wherein the biomarker is AQP5 or is a membrane-bound AQP5.

33.-36. (canceled)

37. A method of i) inhibiting gastric cancer tumorigenesis, ii) gastric cancer progression, or iii) promoting gastric cancer regression, the method comprising administering an AQP5 inhibitor to a subject.

38. (canceled)

39. The method of claim 37, wherein the AQP5 inhibitor inhibits or blocks AQP5 expression and/or function.

40. The method of claim 37, wherein the AQP5 inhibitor is selected from the group consisting of an siRNA, an RNAi, a chimeric antigen receptor (CAR), and a drug.

41.-43. (canceled)

44. The method of claim 40, wherein the drug is selected from the group consisting of enzymatic inhibitors, receptor antagonists, and channel blockers.

45. A method of monitoring progression of a gastric cancer in a subject, the method comprising:
a) measuring the expression level of AQP5 in a sample obtained from the subject after having undergone treatment for gastric cancer; and
b) measuring the expression level of AQP5 in a control sample obtained from the subject prior to treatment for gastric cancer;
wherein an increase in the expression level of AQP5 in the sample of step a compared to the control sample indicates that gastric cancer tumorigenesis has taken place or that the gastric cancer has progressed.

46. A method of monitoring gastric cancer tumorigenesis in a subject, the method comprising:
c) measuring the expression level of AQP5 in a sample obtained from the subject; and
d) measuring the expression level of AQP5 in a reference sample obtained from the subject at a timepoint earlier than the sample of step c;
wherein an increase in the expression level of AQP5 in the sample of step c compared to the reference sample of step d indicates that gastric cancer tumorigenesis has taken place.

47. The method of claim 45, wherein if gastric cancer tumorigenesis has taken place or that the gastric cancer has progressed, the subject is to be treated with an anti-gastric cancer compound.

48.-51. (canceled)


SEQUENCE LISTING (54 sequences)

SEQ ID NO: 1 - DNA, 22 bp, synthetic construct
  source 1..22: mol_type = other DNA
tcctcaccaagcgactccgata

SEQ ID NO: 2 - DNA, 22 bp, synthetic construct
  source 1..22: mol_type = other DNA
acttgagcagcatcgttggcga

SEQ ID NO: 3 - DNA, 23 bp, synthetic construct
  source 1..23: mol_type = other DNA
cagagttctcatttacagccgtc

SEQ ID NO: 4 - DNA, 22 bp, synthetic construct
  source 1..22: mol_type = other DNA
agagaacaggcacgcagtcatg

SEQ ID NO: 5 - DNA, 20 bp, synthetic construct
  source 1..20: mol_type = other DNA
atccgcttgaagagggtggt

SEQ ID NO: 6 - DNA, 21 bp, synthetic construct
  source 1..21: mol_type = other DNA
aggctggaagtggtcataggg

SEQ ID NO: 7 - DNA, 22 bp, synthetic construct
  source 1..22: mol_type = other DNA
tccatgaacccagcccgatctt

SEQ ID NO: 8 - DNA, 22 bp, synthetic construct
  source 1..22: mol_type = other DNA
gaagtagaggattgcagccagg

SEQ ID NO: 9 - DNA, 22 bp, synthetic construct
  source 1..22: mol_type = other DNA
tgagcactgaggaccaggtcaa

SEQ ID NO: 10 - DNA, 23 bp, synthetic construct
  source 1..23: mol_type = other DNA
cgtcagacactaactgcttcacg

SEQ ID NO: 11 - DNA, 22 bp, synthetic construct
  source 1..22: mol_type = other DNA
cctggacattgaatggctgctc

SEQ ID NO: 12 - DNA, 22 bp, synthetic construct
  source 1..22: mol_type = other DNA
gaagcatctcctgccaggaagt

SEQ ID NO: 13 - DNA, 23 bp, synthetic construct
  source 1..23: mol_type = other DNA
gccactattacggacatcttcgg

SEQ ID NO: 14 - DNA, 22 bp, synthetic construct
  source 1..22: mol_type = other DNA
acaacctggtccaactcagcct

SEQ ID NO: 15 - DNA, 23 bp, synthetic construct
  source 1..23: mol_type = other DNA
catcactgccacccagaagactg

SEQ ID NO: 16 - DNA, 23 bp, synthetic construct
  source 1..23: mol_type = other DNA
atgccagtgagcttcccgttcag

SEQ ID NO: 17 - DNA, 22 bp, synthetic construct
  source 1..22: mol_type = other DNA
ccaacgacatcgtcccaggttt

SEQ ID NO: 18 - DNA, 22 bp, synthetic construct
  source 1..22: mol_type = other DNA
ctgcttctcaaaggcactgggt

SEQ ID NO: 19 - DNA, 22 bp, synthetic construct
  source 1..22: mol_type = other DNA
atgcttcaggctgccgtctgtg

SEQ ID NO: 20 - DNA, 22 bp, synthetic construct
  source 1..22: mol_type = other DNA
cagtttccgtcattcggcacca

SEQ ID NO: 21 - DNA, 22 bp, synthetic construct
  source 1..22: mol_type = other DNA
cctgtcggttacctcgtagaga

SEQ ID NO: 22 - DNA, 22 bp, synthetic construct
  source 1..22: mol_type = other DNA
ggactcggaatggctggtttct

SEQ ID NO: 23 - DNA, 20 bp, synthetic construct
  source 1..20: mol_type = other DNA
ggcgttcggtggaggggctt

SEQ ID NO: 24 - DNA, 22 bp, synthetic construct
  source 1..22: mol_type = other DNA
cagatggtggaggaagaaacgg

SEQ ID NO: 25 - DNA, 23 bp, synthetic construct
  source 1..23: mol_type = other DNA
caaaatggcggagaaggcaaagc

SEQ ID NO: 26 - DNA, 22 bp, synthetic construct
  source 1..22: mol_type = other DNA
tcaaagctgcgaggagacggtt

SEQ ID NO: 27 - DNA, 23 bp, synthetic construct
  source 1..23: mol_type = other DNA
gcaataagtcgcctgtgtgaagc

SEQ ID NO: 28 - DNA, 23 bp, synthetic construct
  source 1..23: mol_type = other DNA
catcagtgtgaggctgcttgttg

SEQ ID NO: 29 - DNA, 22 bp, synthetic construct
  source 1..22: mol_type = other DNA
tacggtgtggcaccgctcaatg

SEQ ID NO: 30 - DNA, 22 bp, synthetic construct
  source 1..22: mol_type = other DNA
agtcagtggaggcgaagatgca

SEQ ID NO: 31 - DNA, 22 bp, synthetic construct
  source 1..22: mol_type = other DNA
tagccaggacatgaggcagatc

SEQ ID NO: 32 - DNA, 23 bp, synthetic construct
  source 1..23: mol_type = other DNA
gctttagggattcttgctgctgg

SEQ ID NO: 33 - DNA, 22 bp, synthetic construct
  source 1..22: mol_type = other DNA
gtgacagcagttggcttctcca

SEQ ID NO: 34 - DNA, 22 bp, synthetic construct
  source 1..22: mol_type = other DNA
ggagattgcactggatgtcacc

SEQ ID NO: 35 - DNA, 22 bp, synthetic construct
  source 1..22: mol_type = other DNA
ctggagaggtagtgacaaccac

SEQ ID NO: 36 - DNA, 22 bp, synthetic construct
  source 1..22: mol_type = other DNA
gcagaggattgttgtggtgctg

SEQ ID NO: 37 - DNA, 22 bp, synthetic construct
  source 1..22: mol_type = other DNA
gtctcctctgacttcaacagcg

SEQ ID NO: 38 - DNA, 22 bp, synthetic construct
  source 1..22: mol_type = other DNA
accaccctgttgctgtagccaa

SEQ ID NO: 39 - DNA, 23 bp, synthetic construct
  source 1..23: mol_type = other DNA
tgagaagacttgtgccaactgct

SEQ ID NO: 40 - DNA, 22 bp, synthetic construct
  source 1..22: mol_type = other DNA
ccctgttgcctgaagcatcttc

SEQ ID NO: 41 - DNA, 23 bp, synthetic construct
  source 1..23: mol_type = other DNA
gggtcaatagtcaacgaggagac

SEQ ID NO: 42 - DNA, 22 bp, synthetic construct
  source 1..22: mol_type = other DNA
gccaagtcatctgtggaaggtg

SEQ ID NO: 43 - DNA, 22 bp, synthetic construct
  source 1..22: mol_type = other DNA
ctatgggatgcacagggtggaa

SEQ ID NO: 44 - DNA, 23 bp, synthetic construct
  source 1..23: mol_type = other DNA
cgcaggtgtaaaagacaaggcag

SEQ ID NO: 45 - DNA, 22 bp, synthetic construct
  source 1..22: mol_type = other DNA
acctcctgactacttccagtgc

SEQ ID NO: 46 - DNA, 23 bp, synthetic construct
  source 1..23: mol_type = other DNA
ggaaggtaacctcgatgttggtg

SEQ ID NO: 47 - DNA, 22 bp, synthetic construct
  source 1..22: mol_type = other DNA
gatggcaaccaagtcgtgagag

SEQ ID NO: 48 - DNA, 23 bp, synthetic construct
  source 1..23: mol_type = other DNA
cctggcattgaaggctaatcctc

SEQ ID NO: 49 - RNA, 28 nt, synthetic construct
  source 1..28: mol_type = other RNA
aaaactctgcgaacacggcccctgtctc

SEQ ID NO: 50 - RNA, 28 nt, synthetic construct
  source 1..28: mol_type = other RNA
aaggccgtgttcgcagagttcctgtctc

SEQ ID NO: 51 - RNA, 21 nt, synthetic construct
  source 1..21: mol_type = other RNA
cggtggtcatgaatcggtttt

SEQ ID NO: 52 - RNA, 21 nt, synthetic construct
  source 1..21: mol_type = other RNA
aaccgattcatgaccaccgca

SEQ ID NO: 53 - RNA, 21 nt, synthetic construct
  source 1..21: mol_type = other RNA
gcgtgtggccatcatcaaatt

SEQ ID NO: 54 - RNA, 21 nt, synthetic construct
  source 1..21: mol_type = other RNA
tttgatgatggccacacgctt